(19) United States
(12) Patent Application Publication
(10) Pub. No.: US 2024/0161915 A1
(43) Pub. Date: May 16, 2024

(54) SYSTEM AND METHOD FOR DETERMINING PATIENT ACCESS IN A HEALTHCARE FACILITY

(71) Applicant: KPMG LLP, New York, NY (US)

(72) Inventors: Eduardo Lopez MOTA, Tomball, TX (US); Alex OBENAUF, Gulfport, FL (US); Michael E. FECTEAU, Newberry, FL (US); Saurabh GOYAL, Leander, TX (US)

(21) Appl. No.: 18/511,699

(22) Filed: Nov. 16, 2023

Related U.S. Application Data
(60) Provisional application No. 63/425,728, filed on Nov. 16, 2022.

(51) Int. Cl. G16H 40/20; G16H 50/20

(52) U.S. Cl. CPC G16H 40/20; G16H 50/20

(57) ABSTRACT

A patient access determination system configured to extract health related data from a data source to form extracted health data, generate a plurality of data pipelines for conveying the extracted health data, and store the extracted health data conveyed over one or more of the data pipelines in a data model to form stored health data. The data model includes tables for organizing and storing the extracted health data. The system also determines from at least the patient encounter data forming part of the stored health data a number of lost appointments that can be recovered by the healthcare facility and apply one or more machine learning models to the stored health data to generate predictions therefrom. The system can also generate one or more user interfaces for displaying selected portions of the stored health data and the predictions.

### RELATED APPLICATION

The present application claims priority to U.S. provisional patent application Ser. No. 63/425,728, filed on Nov. 16, 2022, and entitled System And Method For Determining Patient Access In A Healthcare Facility, the contents of which are herein incorporated by reference.

### BACKGROUND OF THE INVENTION

The present application relates to forecasting methods, and more specifically relates to systems and methods for forecasting patient access to a healthcare facility.

In general, healthcare facilities, such as hospitals, rely on the scheduling of patient appointments and other types of healthcare related services to drive revenue to the facility. Patient scheduling can be as important as the expertise of the healthcare providers (e.g., doctors) who treat the patients in terms of generating revenue for the healthcare facility. As such, accurate patient scheduling and the conversion of patient appointments into billable services is an essential revenue source for the healthcare facility.

Like any other potential revenue source, patient demands and needs are typically prioritized. Indeed, the fiscal health of the healthcare facility can depend on how well the facility manages the overall patient experience, reduces the occurrence of missed appointments, and keeps the patient waiting time as low as possible.

Patient access typically relates to the availability of healthcare related services and the ability of consumers to access health related care and treatment. As such, patient access can include the delivery of healthcare related services, including for example appointment scheduling, registration, financial clearance and related services, charge capture, clinical documentation, medical coding, case management, utilization review for treatments and other healthcare related functions. As such, patient access is a core function of the revenue generation cycle of the healthcare facility, and typically starts with registration, appointment scheduling and all of the related support processes to patients, providers, and payers throughout the patient's healthcare experience. A main purpose of the patient access to the healthcare facility is to drive revenue and to supply or accumulate information which results in building the foundation for medical records, billing, and collections.

As such, conventional techniques employed by healthcare facilities in an attempt to capture revenue by addressing or attempting to reduce missed or canceled patient appointments has several drawbacks. For example, the healthcare facilities often resort to overbooking appointments to minimize missed appointments. However, this can lead to overcrowded waiting rooms, long wait times for patients, and rushed consultations, thus compromising the quality of care and patient experience. Further, an overemphasis on revenue maximization may lead to a focus on quantity over quality. Healthcare providers may prioritize seeing more patients in a day, resulting in less time for each patient, reduced attention to individual cases, and potentially suboptimal healthcare outcomes. As such, patients may become dissatisfied if they perceive a lack of personal attention or long wait times. This can result in a negative patient experience, reduced patient loyalty, and potentially fewer referrals to the facility. Still further, healthcare providers and staff, including doctors, nurses, and administrative personnel, can experience burnout when dealing with a constant stream of patients and high-pressure environments. This can result in high turnover, decreased morale, and reduced job satisfaction.

### SUMMARY OF THE INVENTION

It is an object of the present invention to reduce the incidence of lost appointments (e.g., missed or canceled appointments) and to increase the number of patient appointments so as to enhance the revenue generated by the healthcare facility.

The patient access determination system of the present invention helps to identify, measure, and quantify overall patient access to a healthcare facility, determine a provider's schedule, determine appointment related information including appointment utilization and the like, and process referral opportunities in a single integrated system, thus providing the healthcare facility with the ability to identify areas of economic opportunity in the healthcare facility when considering all aspects of the operation of the healthcare facility.

The present invention is directed to a computer-implemented patient access determination system having a processor and a non-transitory memory having instructions configuring the processor to: extract health related data from a data source using an extract, transform and load technique to form extracted health data, wherein the health data includes patient encounter data, lost appointment data, and schedule data; generate a plurality of data pipelines with the extract, transform and load technique for conveying the extracted health data; store at least a portion of the extracted health data conveyed over one or more of the plurality of data pipelines in a data model to form stored health data, wherein the data model includes a plurality of tables for organizing and storing the extracted health data; determine from at least the patient encounter data forming part of the stored health data a number of lost appointments that can be recovered by the healthcare facility; apply one or more machine learning models to the stored health data to generate predictions therefrom; and generate one or more user interfaces for displaying selected portions of the stored health data and the predictions.

The processor can also be configured to, when determining from the health data stored in the data model a number of lost appointments that can be recovered by the healthcare facility, categorize the patient encounter data and the lost appointment data into a plurality of appointment categories and generate categorization data based thereon, and determine based on the categorization data a number of recoverable appointments from the lost appointments, where the lost appointments were lost based on manageable reasons. The processor is further configured to determine, based on the lost appointment data in the categorization data, one or more appointment parameters associated with the lost appointments, and to determine a recovery factor based on the lost appointment data in the categorization data, where the lost appointments were lost for manageable reasons. The system can be configured to apply the recovery factor to each category of lost appointments to determine the number of recoverable appointments from the lost appointments, and to apply a prestored reimbursement rate dynamic factor to the lost appointment data to determine a reimbursement amount based on the number of lost appointments.

The processor can be still further configured to determine, based on the categorization data, a lost opportunity associated with the lost appointments that are lost for non-recoverable reasons. The system can also determine an access opportunity based on the stored health data indicative of the availability of patient appointments at a selected healthcare facility. The system can also categorize the lost appointment data into a plurality of different categories with a categorization unit configured to generate categorization data which includes lost appointment data, and determine, based on the categorization data, a lost opportunity associated with the lost appointments that are lost for non-recoverable reasons. The plurality of different categories includes a first category for storing non-recoverable canceled appointment data from the lost appointment data and a second category for storing patient non-recoverable canceled appointment data from the lost appointment data. The system can then apply a recovery factor to each of the plurality of categories to determine a number of recovered appointments and apply a reimbursement rate to determine revenue associated with the number of recovered appointments.

The processor is further configured to determine from the stored health data an access opportunity that the patient has access to appointments available at a selected healthcare facility. The processor is further configured to train the machine learning model with the health data including lost appointment data to form a trained machine learning model, and tune the trained machine learning model to perform one or more selected tasks by training the machine learning model on a narrower dataset of the health data and adjusting one or more tuning parameters to perform the selected task.

Still further, the processor can be configured to generate the plurality of data pipelines. The data pipelines can include one or more or, two or more of, three or more of, or four or more of: a patient encounter standardization pipeline to convey patient encounter data and to standardize the patient encounter data using a standardization technique; a temporal master file creation pipeline to convey one or more temporal master files based on the patient encounter data and from provider schedule data; a final master file creation pipeline to convey a final master file based on the temporal master files; a file normalization pipeline to normalize and standardize one or more files associated with the patient encounter data and provider schedule data; a patient encounter final output pipeline to create and convey a final patient encounter output; and a provider schedule final output pipeline to create and convey a final provider schedule output. The common data model can include a plurality of data tables, including a patient encounter table for storing patient encounter data, and a provider schedule table for storing provider schedule data associated with schedules of one or more healthcare providers associated with a healthcare facility. The common data model an also include either one or more of, two or more of, three or more of, or four or more of: an appointment language table for storing information associated with a language of the patient or healthcare provider; a provider specialty table for storing information associated with a specialty of the healthcare provider in the healthcare facility; a provider schedule-session length table for storing data associated with a length of an appointment; a provider schedule-session per day table for storing data associated with a number of appointments per day at the healthcare facility; appointment unavailability table for storing information associated with dates that appointments are not available in the schedule of the healthcare provider or the healthcare facility and reasons for unavailability of the appointment; a time held table for storing information associated with time held in a schedule of the healthcare provider or the healthcare facility and one or more reasons that the time is held; a day held table for storing information associated with a day held in a schedule of the healthcare provider or the healthcare facility and one or more reasons that the day is held; a time unavailable table for storing information associated with a time that is unavailable in a schedule of the healthcare provider or the healthcare facility and associated appointment classification information; a chronic diagnosis table for storing diagnostic information associated with a patient and any related healthcare code information; an appointment type table for storing information associated with a type of appointment and date information associated with the appointment; a last appointment table for storing information associated with a last appointment of the patient, including time since last appointment and appointment cancellation information; an appointment schedulers table for storing information associated with a person in the healthcare facility scheduling a patient appointment including person identification information and provider specialty group information; an appointment cancellation reason table for storing information associated with a reasons that a patient appointment was cancelled; an encounter type table for storing information associated with a type of patient encounter; a location type table for storing information associated with a location of the healthcare provider; and an appointment lag table for storing information associated an appointment lag data. The patient encounter table stores appointment data including appointment date and time, reason for appointment, appointment cancellation data, status data, appointment specific codes associated with the healthcare facility, and appointment billing information. The provider schedule table stores schedule data including provider availability data, appointment start and stop time data, appointment date data, healthcare provider classification data, and a number of available appointments at the healthcare facility. The provider specialty table stores identification information of the healthcare provider.

The present invention can also be directed to a computer-implemented method for determining patient access to a healthcare facility, comprising extracting health related data from a data source using an extract, transform and load technique to form extracted health data, wherein the health data includes patient encounter data, lost appointment data, and schedule data; generating a plurality of data pipelines with the extract, transform and load technique for conveying the extracted health data; storing at least a portion of the extracted health data conveyed over one or more of the plurality of data pipelines in a data model to form stored health data, wherein the data model includes a plurality of tables for organizing and storing the extracted health data; determining from at least the patient encounter data forming part of the health data stored a number of lost appointments that can be recovered by the healthcare facility; applying one or more machine learning models to the stored health data to generate predictions therefrom; and generating one or more user interfaces for displaying selected portions of the stored health data and the predictions.

The step of determining from the health data stored in the data model a number of lost appointments that can be recovered by the healthcare facility comprises categorizing the patient encounter data and the lost appointment data into a plurality of appointment categories and generate categorization data based thereon, and determining based on the categorization data a number of recoverable appointments from the lost appointments, wherein the lost appointments were lost based on manageable reasons. The method can also optionally include one or more of determining, based on the lost appointment data in the categorization data, one or more appointment parameters associated with the lost appointments; determining a recovery factor based on the lost appointment data in the categorization data, where the lost appointments were lost for manageable reasons; applying a recovery factor to each category of lost appointments so as to determine the number of recoverable appointments from the lost appointments; and applying a prestored reimbursement rate dynamic factor to the lost appointment data to determine a reimbursement amount based on the number of lost appointments.

The method of the present invention can also include determining, based on the categorization data, a lost opportunity associated with the lost appointments that are lost for non-recoverable reasons, or determining from the stored health data an access opportunity that the patient has to appointments available at a selected healthcare facility.

The method of the present invention can further include categorizing the lost appointment data into a plurality of different categories, wherein the categorization unit generates categorization data which includes lost appointment data, and determining, based on the categorization data, a lost opportunity associated with the lost appointments that are lost for non-recoverable reasons. The plurality of different categories includes a first category for storing non-recoverable canceled appointment data from the lost appointment data and a second category for storing patient non-recoverable canceled appointment data from the lost appointment data. The method includes applying a recovery factor to each of the plurality of categories to determine a number of recovered appointments and applying a reimbursement rate to determine revenue associated with the number of recovered appointments. The method also includes determining from the stored health data an access opportunity that the patient has to appointments available at a selected healthcare facility.

The method of the present invention also contemplates and includes training the machine learning model with the health data including lost appointment data to form a trained machine learning model and tuning the trained machine learning model to perform one or more selected tasks by training the machine learning model on a narrower dataset of the health data and adjusting one or more tuning parameters to perform the selected task.

The computer-implemented method further comprises generating one or more of a patient encounter standardization pipeline to convey patient encounter data and to standardize the patient encounter data using a standardization technique; a temporal master file creation pipeline to convey one or more temporal master files based on the patient encounter data and from provider schedule data; a final master file creation pipeline to convey a final master file based on the temporal master files; a file normalization pipeline to normalize and standardize one or more files associated with the patient encounter data and provider schedule data; a patient encounter final output pipeline to create and convey a final patient encounter output; and a provider schedule final output pipeline to create and convey a final provider schedule output.

The common data model can include one or more of a patient encounter table for storing patient encounter data, and a provider schedule table for storing provider schedule data associated with schedules of one or more healthcare providers associated with a healthcare facility. The common data model can further include one or more of, two or more of, three or more of, or four or more of: an appointment language table for storing information associated with a language of the patient or healthcare provider; a provider specialty table for storing information associated with a specialty of the healthcare provider in the healthcare facility; a provider schedule-session length table for storing data associated with a length of an appointment; a provider schedule-session per day table for storing data associated with a number of appointments per day at the healthcare facility; an appointment unavailability table for storing information associated with dates that appointments are not available in the schedule of the healthcare provider or the healthcare facility and reasons for unavailability of the appointment; a time held table for storing information associated with time held in a schedule of the healthcare provider or the healthcare facility and one or more reasons that the time is held; a day held table for storing information associated with a day held in a schedule of the healthcare provider or the healthcare facility and one or more reasons that the day is held; a time unavailable table for storing information associated with a time that is unavailable in a schedule of the healthcare provider or the healthcare facility and associated appointment classification information; a chronic diagnosis table for storing diagnostic information associated with a patient and any related healthcare code information; an appointment type table for storing information associated with a type of appointment and date information associated with the appointment; a last appointment table for storing information associated with a last appointment of the patient, including time since last appointment and appointment cancellation information; an appointment schedulers table for storing information associated with a person in the healthcare facility scheduling a patient appointment including person identification information and provider specialty group information; an appointment cancellation reason table for storing information associated with a reasons that a patient appointment was cancelled; an encounter type table for storing information associated with a type of patient encounter; a location type table for storing information associated with a location of the healthcare provider; and an appointment lag table for storing information associated an appointment lag data.

According to another embodiment, the present invention is directed to a patient access determination system for determining patient access to a healthcare facility comprising a data source for providing health related data, wherein the health-related data includes patient encounter data, scheduling data, and lost appointment data; an extraction unit for extracting the health-related data from the data source using an extract, transform and load technique to form extracted health data; a patient encounter determination unit for receiving the extracted health data and for storing a portion of the extracted health data in a data model to form stored health data and for determining based on the lost appointment data in the stored health data a number of recoverable appointments from the lost appointment data; a prediction unit for receiving the stored health data from the data model and applying one or more machine learning techniques to the stored health data to generate insights and predictions therefrom; and a reporting unit for generating one or more user interfaces for displaying information associated with the insights and predictions.

The patient encounter determination unit comprises a demand opportunity determination unit for processing the stored health data and determining a demand opportunity based on the stored health data. The demand opportunity determination unit can include a categorization unit for categorizing the stored health data including the lost appointment data into a plurality of categories and for generating categorization data associated therewith, and an appointment recovery determination unit for determining based on the categorization data the number of recoverable appointments from the lost appointments, wherein the lost appointments were lost based on manageable reasons, and for generating recoverable appointment data associated therewith. The demand opportunity determination unit can further include an appointment parameter determination unit for determining, based on the lost appointment data in the categorization data, one or more appointment parameters associated with the lost appointments. The appointment parameters include a length of an appointment based on a specialty of a healthcare provider and a historical appointment length. The appointment parameter determination unit is optionally configured to determine a recovery factor based on canceled appointment data forming part of the lost appointment data in the categorization data, and apply the recovery factor to each category of lost appointments so as to determine the number of recoverable appointments from the lost appointments, or apply a prestored reimbursement rate dynamic factor to the lost appointment data to determine a reimbursement amount based on the number of lost appointments.

The patient encounter determination unit further includes a lost patients opportunity determination unit for determining a lost patients opportunity based on the lost appointment data. The lost patients opportunity determination unit can include a categorization unit for categorizing the lost appointment data into a plurality of different categories where the categorization unit generates categorization data which includes lost appointment data, and a lost opportunity determination unit for determining, based on categorization data, the lost patients opportunity associated with the lost appointments that are lost for non-recoverable reasons. The categorization unit employs a first category for storing non-recoverable canceled appointment data from the lost appointment data and a second category for storing patient non-recoverable canceled appointment data from the lost appointment data. The lost patients opportunity determination unit applies a recovery factor to each of the categories to determine the number of recovered appointments, and then the lost opportunity determination unit applies a reimbursement rate to the number of recovered appointments to determine the revenue associated with the recovered appointments.

The patient encounter determination unit further comprises an access opportunity determination unit for determining from the stored health data an access opportunity that the patient has to appointments available at the healthcare facility, and/or an optimization opportunity determination unit for determining, based on the stored health data including appointment data, an optimization opportunity associated with optimizing an unavailable appointment time portion of the appointment data of the healthcare facility. The optimization opportunity determination unit comprises a categorization unit for categorizing an unavailable appointment time portion of the appointment data into a plurality of categories, and an opportunity determination unit for determining a number of appointments that are recoverable from the unavailable appointment times. The plurality of categories includes a first category for storing an appropriate unavailable appointment time data, and a second category for storing an inappropriate unavailable appointment time category.

The opportunity determination unit is configured to identify a total recoverable appointment time that is recoverable from the unavailable appointment time, determine a number of potential available appointments by applying a preselected appointment time length to the total recoverable appointment time, apply a recovery factor to the number of potential available appointments to determine a number of actual available appointment time, and apply a reimbursement rate to the number of actual available appointment times to determine revenue generated by the actual available appointment times.

The reporting unit generates a first user interface in response to the stored health data for displaying the appointment data, wherein the first user interface comprises a window having a first pane element disposed on a left hand side of the window and second and third vertically stacked pane elements disposed on a right hand side of the window, wherein the first pane element is configured to display lost appointment data. The first pane element includes a first display element having a first graphical element for displaying the lost appointment data and a table element for displaying and organizing the lost appointment data in a tabular format, and wherein the second pane element includes a second display element having a second graphical element for displaying the lost appointment data and the recoverable appointments data. The third pane element includes a third display element having a third graphical element that displays the recoverable appointments by specialty practice in the healthcare facility.

### BRIEF DESCRIPTION OF THE FIGURES

These and other features and advantages of the present invention will be more fully understood by reference to the following detailed description in conjunction with the attached drawings, in which like reference numerals refer to like elements throughout the different views. The drawings illustrate principals of the invention and, although not to scale, show relative dimensions.

FIG. 1 is a schematic block diagram of the patient access determination system according to the teachings of the present invention.

FIG. 2 is a schematic diagram of the patient encounter determination unit of the patient access determination system of the present invention.

FIG. 3 is a schematic representation of a data model employed by the patient encounter determination unit according to the teachings of the present invention.

FIG. 4A is a schematic representation of the specifics of the demand opportunity determination unit of the patient encounter determination unit of the present invention.

FIG. 4B is a schematic representation of the specifics of the lost patients opportunity determination unit of the patient encounter determination unit of the present invention.

FIG. 4C is a schematic representation of the specifics of the optimization opportunity determination unit of the patient encounter determination unit of the present invention.

FIG. 5 illustrates a user interface generated by a user interface generator of the reporting unit showing the associated pane elements of a generated window and the general demand opportunity information that can be displayed within the pane elements.

FIG. 6 illustrates a user interface generated by a user interface generator of the reporting unit showing the associated pane elements of a generated window and the general lost patients opportunity information that can be displayed within the pane elements.

FIG. 7 illustrates a user interface generated by a user interface generator of the reporting unit showing the associated pane elements of a generated window and the general access opportunity information that can be displayed within the pane elements.

FIG. 8 illustrates a user interface generated by a user interface generator of the reporting unit showing the associated pane elements of a generated window and the general optimization opportunity information that can be displayed within the pane elements.

FIG. 9 is a schematic diagram of an electronic or computing device and/or associated system suitable for implementing the patient access determination system of the present invention.

### DETAILED DESCRIPTION

The present invention is directed to a system and method for automatically determining patient encounter, patient access and lost appointment opportunities in an institution, such as a healthcare facility, based on a number of selected health related parameters. The health-related parameters or data can be processed in a specific manner by a computer implemented system and method so as to enable the system to determine selected types of important appointment related information for the healthcare facility. The information an include determining the impact of recovering lost patient appointments (e.g., demand opportunity), of keeping or maintaining lost patient appointments (e.g., lost patients opportunity), of determining actual available time to schedule patient appointments (e.g., access opportunity), and the ability to optimize manageable unavailable time in the healthcare facility in order to convert at least a portion of this time into patient appointments (e.g., optimization opportunity).

As used herein, the term “institution” is intended to include any type of company, enterprise, facility, or organization that has employees and includes one or more physical plants. The institution can include, according to one example, a healthcare facility.

As used herein, the term “healthcare facility” is intended to include hospitals, clinics, facilities rendering medical and healthcare related services, clinician offices, and the like.

As used herein, the term “healthcare provider” or “provider” is intended to include any person associated with the delivery of healthcare related services to a patient. Examples of providers include, doctors, nurses, physician assistants, medical assistants, medical coders, general assistants, people associated with billing, accounting, registration, and scheduling, and the like.

As used herein, the term “health data” or “health-related data” includes any type of data related to the scheduling, delivery, and application of healthcare related services to a person, such as a patient, and to healthcare related claims and associated billing information. Examples of suitable types of data include patient encounter data (e.g., appointment data and schedule data), medical data, registration data, demographic data, psychological and mental related data, medication related data, radiological data, test and laboratory result data, dental related data, disease related data, medical provider data including the type of healthcare provider, prescription data, immunization data, genetics related data, body measurement related data (e.g., height, weight, blood pressure, and the like), referral related data, climate and pollution or emission related data, insurance related data, billing data, information created or generated by healthcare professionals, data from monitoring devices such as wearable and non-wearable devices, revenue data associated with the delivery of health services, and the like. The health-related data can be provided in any selected form or format and can be stored in any type of storage medium and format and is typically provided as part of an electronic health record.

As used herein, the term “patient encounter’ and associated data is intended to mean an interaction in a healthcare facility in which a patient can interact with a healthcare provider where the healthcare provider delivers health related services to the patient, including, without limitation, admission data, appointment data and other types of scheduling data, treatment data, performance or interpretation of diagnostic tests, or consultations, and can include the supervision of staff of the healthcare facility. The patient encounter data can include or can be related to, in addition to appointment data and schedule data, demand opportunity data, lost patient opportunity data, access opportunity data, and optimization opportunity data.

As used herein, the term “electronic health record” or (EHR) is intended to refer to a digital record of health-related data associated with a patient. The electronic health record can be stored in any selected location, such as at a healthcare facility. The record can refer to a systematized collection of patient and population health related data or information that is stored in a digital format. The records can be shared across different health care settings. The records can be shared through network-connected, enterprise-wide information systems or other information networks and exchanges.

As used herein, the term “appointment” or patient appointment” is intended to refer to or mean a scheduled and pre-arranged meeting between a patient and a healthcare provider, such as a physician, physician assistant, nurse, specialist, or other medical professional, for the purpose of receiving medical care, evaluation, treatment, or consultation. The appointment can be scheduled as a slot or session in scheduling application employed by the healthcare facility. Patient appointments are one or many selected aspects of healthcare delivery, facilitating the efficient provision of healthcare services while ensuring that patients receive timely and appropriate care.

As used herein, the term “lost appointment” refers to appointments typically conducted in a healthcare facility that have been either missed by the patient or healthcare facility and/or canceled by the patient or by the healthcare facility, resulting in a slot or opening in a schedule of a healthcare provider in the healthcare facility that could have been used for providing care to another patient or to the scheduled patent. The lost appointments can include different types of appointments, including for example canceled appointments and missed appointments. The missed appointments can correspond to appointments where a patient fails to show up without prior notice or cancelation or leaves the facility without being seen by a healthcare provider or can correspond to appointments that were missed by the healthcare provider. The healthcare facility and the healthcare provider can set aside time and resources for the appointment but does not generate any revenue from the scheduled appointment when the appointment is missed. The canceled appointments can correspond to appointments that the patients have scheduled but later cancel before or at the scheduled appointment time. While the patient may not physically attend the appointment, the patient typically gives advance notice of their intention to cancel. However, if the cancellation occurs too close to the appointment time, it may not provide the healthcare provider with adequate time to fill that slot with another patient, resulting in a lost appointment and associated lost revenue. Alternatively, the canceled appointment can be directed to an appointment that was canceled by the healthcare provider. In both cases, missed and canceled patient appointments represent a lost opportunity for healthcare providers to deliver care, generate revenue, and optimize their scheduling and resource allocation. Managing and reducing the number of lost patient appointments can help the healthcare facility maximize operational efficiency and revenue while ensuring patients receive timely and quality care.

As used herein, the term “schedule data” or “provider schedule data” is intended to refer to or mean structured and organized information or data that outlines or is directed to the planned work schedule, availability, and appointments of patients and of healthcare providers, such as physicians, physician assistants, nurses, specialists, and other clinical professionals. The data can be employed to help efficiently manage the scheduling and allocation of healthcare services and healthcare provider resources, optimize resource utilization, and ensure that patients receive timely and appropriate care. The data can include, for example, provider information including names, roles, specialties, and contact information, and scheduling information which includes dates, times, and duration of provider work shifts, as well as any recurring schedules or on-call duties. The data can also include appointment information about the availability of providers for patient appointments, including the types of appointments that can be accommodates (e.g., new patient visits, follow-up appointments, procedures), and the number of appointment slots or sessions available for each type. The data can further include patient appointment data including a record of scheduled patient appointments, including patient names, appointment times, and the providers assigned to each appointment; healthcare facility location data including information about the specific healthcare facility or clinic where the provider is scheduled to work; provider availability and absence information including vacation time, time off, and any unexpected absences or schedule changes. The data can still further include provider special request data including any specific requests or preferences made by the providers, such as preferences for certain days off, preferred shift times, or specific procedures they can or cannot perform. The data can also include scheduling rules and policy data related to provider scheduling, such as maximum work hours, minimum rest periods between shifts, and adherence to regulatory compliance (e.g., labor laws). The provider schedule data helps the healthcare facilities effectively allocate clinical resources, reduce wait times for patients, and ensure that the right provider is available for the right patient at the right time.

The term “graphical user interface” or “user interface” as used herein refers to any software application or program, which is used to present data to an operator or end user via any selected hardware device, including a display screen, or which is used to acquire data from an operator or end user for display on the display screen. The interface can be a series or system of interactive visual components that can be executed by suitable software. The user interface can hence include screens, windows, frames, panes, forms, reports, pages, buttons, icons, objects, menus, tab elements, and other types of graphical elements that convey or display information, execute commands, and represent actions that can be taken by the user. The objects can remain static or can change or vary when the user interacts with them.

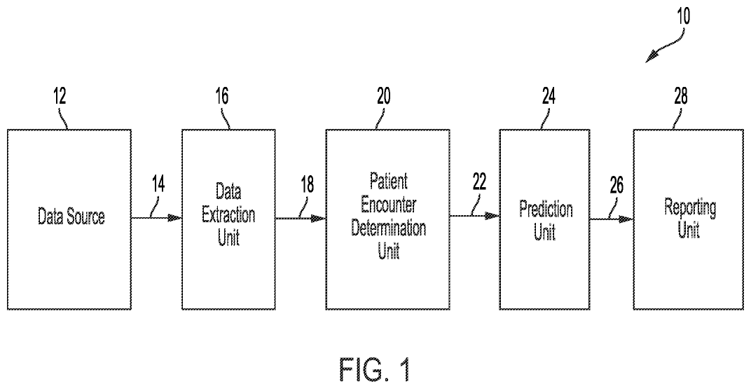

The present invention is directed to patient access determination system as shown for example in FIG. 1. The illustrated patient access determination system 10 can be configured to assess, determine, or forecast the ability of a patient to access medical services in an institution, such as a healthcare facility. The illustrated patient access determination system 10 can include one or more data sources 12 for providing health-related data 14 to the system. For the sake of simplicity, the data source 12 is shown as a single unit, although the health-related data can be provided by or stored in multiple different data sources disposed at one or more discrete locations. For example, the health-related data or portions thereof can be provided by way of an electronic health record. The health-related data can also be provided by the patient or by the healthcare facility or both. According to one embodiment, the health-related data 14 from the data source 12 can include patient encounter data, provider schedule data, appointment data, lost appointment data, provider referral related data, registration data, schedule data, and the like.

The health-related data 14 can be extracted from the data source 12 by the data extraction unit 16. The data extraction unit 16 can employ one or more extraction, transform and load (ETL) techniques for extracting the data from the data source and for ingesting the data. Specifically, the ETL technique extracts the heath related data 14 from the data source 12 and ingests the health-related data 14 by cleaning the data and transforming the data into a consistent data format, and then loading the extracted health related data 18 into a patient encounter determination unit 20. An example of a suitable ETL technique is the ETL process from Alteryx, USA. The ETL technique can form, create, or generate one or more data pipelines or data workflows between the data source 12 and the patient encounter determination unit 20. The purpose of the data pipelines is to be able to automate the flow of data from different input data sources 12 to a specific system or systems that require the data. According to one embodiment, the ETL technique can implement or create one or more data pipelines or a series of data pipelines or workflows between the data extraction unit 16 and the patient encounter determination unit 20, generally illustrated as the extracted health-related data 18. The data pipelines, and particularly the specific data pipelines of the present invention, improve the processing capabilities of the underlying computing infrastructure and enhance the ability to process, store, and manipulate the data in real time. The data pipelines can include for example one or more of a patient encounter standardization pipeline, a provider schedule standardization pipeline, a temporal master file creation pipeline, a final master file creation pipeline, a source files normalization pipeline, a patient encounter final output pipeline, and a provider schedule final output pipeline. The extracted health-related data 18 flowing over the data pipelines can be extracted according to any selected sequence, including in series, in parallel, or a combination of both. According to one embodiment, the health-related data 18 is extracted in sequence over multiple ones of the foregoing data pipelines.

The patient encounter standardization data pipeline can be used to extract and convey patient encounter data and to standardize the patient encounter data. The data pipeline allows for the identification of possible data conflicts (e.g., duplications, invalid records, and the like) and updates the formats of the patient encounter data to the formats required by the patient encounter determination unit 20. The provider schedule standardization data pipeline can be used to standardize provider schedule data and allows for the identification of possible data conflicts and updates the provider schedule data formats to the formats required by the patient encounter determination unit 20. The temporal master file creation data pipeline creates temporal master files from the patient encounter data and from the provider schedule data that includes data associated with one or more of healthcare practice specialties, payors, appointment types, appointment statuses, and appointment cancelation reasons. The temporal master files can refer to files or data structures that store historical or time-related information about master data, which can include information about patients, providers, facilities, and other core entities. The temporal master files can store historical versions of the master data, thus allowing for the analysis of changes and trends over time. The temporal master files can also include unique values for each of the fields using original identification (ID) information from the healthcare provider systems. The master data files can be created during the ETL process by extracting data from the data source 12, transforming the data to include relevant historical information, and then loading the data into the temporal master files. The final master files creation data pipeline can create or generate a final master file for all of the required categories including the ones with the temporal master file. The final master files creation data pipeline can employ one or more mapping techniques that creates category fields for the files with temporal masters and can include the new fields into the fields of the final master file. The ETL process can also create a new ID for each of the values on each of the master files.

The source files normalization data pipeline can employ the master files to normalize and standardize the files and associated data structure associated with the patient encounter data and the provider schedule data such that the data is consistent and ready for subsequent processing and analysis. The standardization can include replacing the original ID values for the ones created in the previous workflow and deleting the unnecessary description columns for each of the fields that have a master file, reducing the size of the file and preparing the encounter and provider schedule data files to be used in the data model. The patient encounter final output data pipeline creates the final patient encounter output and adds to the data set the required information to identify the patient's last encounter and all other necessary fields to be used by a reporting unit 28. The provider schedule final output data pipeline creates the final provider schedule output and adds to the data set the required information to determine or calculate patient appointment or session length and other key data elements for subsequent use by the reporting unit 28.

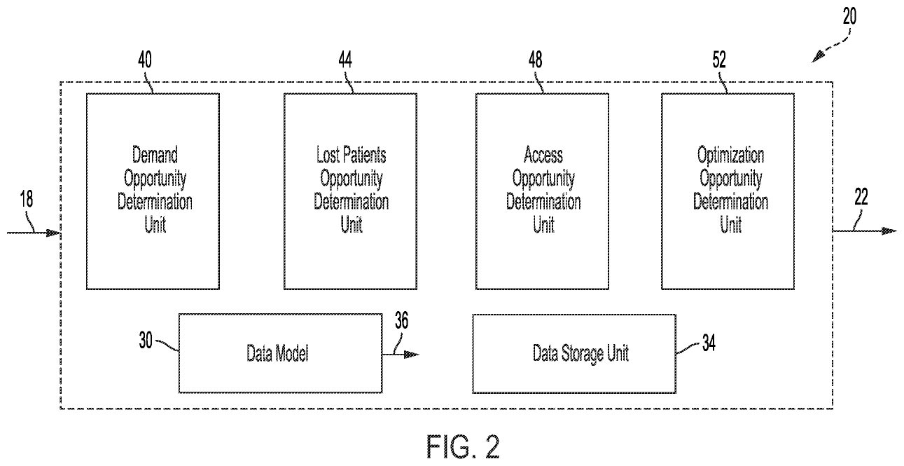

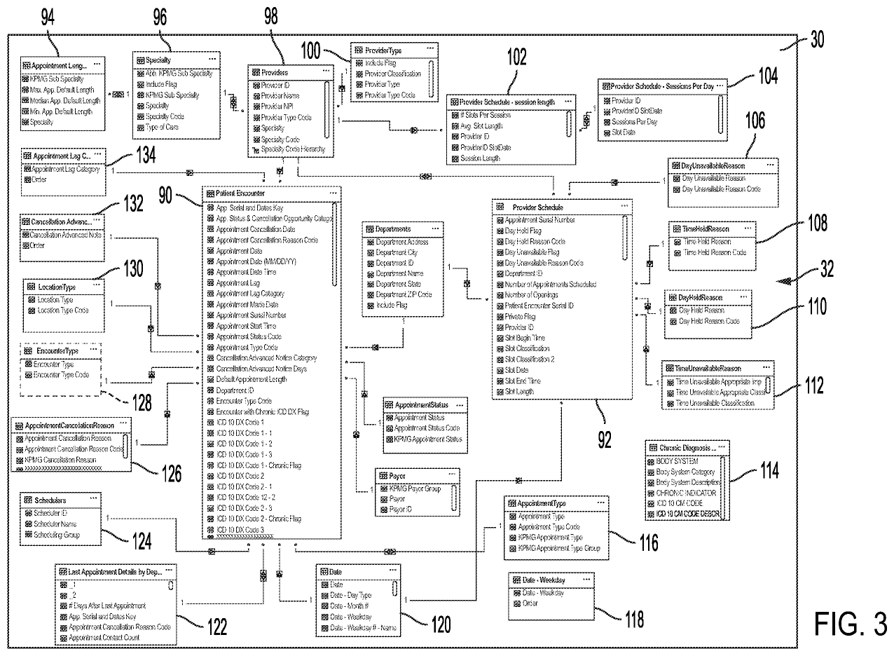

The extracted health-related data 18 provided, for example, through one or more of the data pipelines can then be stored in a data model, such as in the data model 30 illustrated in FIGS. 2 and 3. The data model 30 can form part of the patient encounter determination unit 20 or can be separate therefrom and can be stored in the data storage unit 34 or can be separately stored. The data model 30 can include a set of tables 32 for organizing and storing the extracted health-related data 18. The tables 32 can be constructed or organized in a tabular format with rows and columns, where each row can for example represent a record or data entity and each column can represent an attribute or field of the data entity. According to one embodiment, the set of tables 32 of the data model 30 can include a patient encounter table 90 for storing various types of patient encounter data. For example, the table 90 can set forth data including one or more of appointment specific data including appointment date and time, reason for appointment, appointment cancellation data, appointment status data, appointment specific codes associated with the healthcare facility, appointment billing information, and the like. The tables 32 can also include a provider schedule table 92 for storing provider schedule data associated with the schedules of one or more healthcare providers associated with the healthcare facility. The provider schedule data that is stored in the provider schedule table can include schedule specific information, such as provider availability, dates, appointment start and stop times, provider classification data, appointment numbers including number of available appointments, and other types of appointment related information and provider related information. The patient encounter table 90 and the provider schedule table 92 can function as the main, core or central tables in the data model 30. The main tables 90, 92 store and hold fundamental information in the data model 30 and are often at the center of data analysis and processing.

The illustrated data model 30 can also include non-core or auxiliary tables that help or assist with data management, data integration, and system behavior. The auxiliary tables can include for example one or more of lookup tables, configuration tables, audit or log tables, user or security tables, metadata tables, utility tables, historical tables, and the like. The lookup tables can store reference data, such as codes, categories, or lists of values, used in other tables to maintain data consistency, so as to avoid data redundancy. As such, the lookup tables help standardize and validate data across the data model 30. The configuration tables can store settings, preferences, or parameters that customize the behavior of the system. The audit tables can be used to capture and record changes, actions, or events that occur within the system, thus offering an audit trail and history of changes in the data. The user and security tables can be sued for user authentication and authorization, managing user profiles, and defining access permissions to control who can access, modify, or delete data in the data model 30. The metadata tables can store information about the data model 30, including table and column descriptions, data types, relationships, and data lineage. The utility tables can serve various auxiliary functions, such as generating unique identifiers (e.g., sequence numbers), providing date-time reference data, or managing system-specific information, and can help ensure data consistency. The historical tables store historical versions of data, which can be useful for tracking changes over time and historical analysis.

As illustrated in the data model 30, the tables 32 can further include one or more of the following auxiliary tables, including an appointment length table 94 that sets forth information associated with the provider appointment length by patient for each healthcare provider and specialty practice or group; a provider specialty table 98 that sets forth data or information associated with the specialties of the healthcare providers in the healthcare facility, including healthcare provider identification information including name, identification information, type of specialty, specialty healthcare codes, and the like; a provider type table 100 that sets forth healthcare provider type and classification information; a provider schedule-session length table 102 that includes data associated with the appointment or session length, average session length, and other types of provider related information; a provider schedule-session per day table 104 that includes data associated with sessions per day, session information, and the like; appointment unavailability table 106 that includes information associated with the dates that appointments are not available in the schedule of the provider and/or the healthcare facility, reasons for the unavailability, and the like; a time held table 108 that includes information associated with the time held in a schedule of the provider or the healthcare facility, the reasons that the time is held and any relevant reason codes, and the like; a day held table 110 that includes information associated with the day held in a schedule of the healthcare provider or the healthcare facility, the reasons that the day is being held and any relevant reason codes, and the like; a time unavailable table 112 that includes information associated with the time that is unavailable in a schedule, any associated classification information, and the like; a chronic diagnosis table 114 that includes diagnostic information associated with patient and any related healthcare code information; an appointment type table 116 that includes information associated with the type of appointment and related codes; date tables 118 and 120 that include date information associated with the appointment; last appointment table 122 that includes information associated with the last appointment of the patient, including time since last appointment, appointment cancellation information, and the like; an appointment schedulers table 124 that includes information associated with the person scheduling the patient appointment including identification information, provider specialty group information, and the like; an appointment cancellation reason table 126 that includes information associated with the reasons that the appointment was cancelled, associated code information, and the like; an encounter type table 128 that includes information associated with the type of patient encounter; a location type table 130 that includes information associated with the type of location of the healthcare provider or specialty group, associated code information, and the like; and an appointment lag table 132 that includes appointment lag data, which refers to the time delay or gap between when a patient requests an appointment with a healthcare provider and the actual appointment date. [Please confirm the brief descriptions of the tables in the data model. Please provide any other relevant information about the tables and data model that you deem important to provide. In the auxiliary tables, are any of the tables more important than other tables?] Those of ordinary skill in the art will readily recognize that the data model 30 can be constructed and configured in a way that can be easily expanded by the addition of other relevant data relative to the patient appointments, lost appointments, provider schedule, or any other activity related to patient care. For example, additional tables can be employed that can store data related to clinical full-time employees (cFTEs) or decision trees used during the scheduling process. This type of flexibility allows the data model 30 to be flexible and can accommodate customized solutions.

The stored health data 22 that is stored in the data model 30 and processed by the patient encounter determination unit 20 can be conveyed to an optional prediction unit 24 for applying to the stored health-related data 22 one or more types of machine learning models or techniques. As used herein, the term “machine learning technique” or “machine learning model” is intended to mean the application of one or more software application techniques or models that process and analyze data to draw inferences, insights and/or predictions from patterns in the data. The machine learning techniques can include a variety of models or algorithms, including supervised learning models, unsupervised learning models, reinforcement learning models, knowledge-based learning models, natural-language-based learning models such as natural language generation and natural language processing, deep learning models, and the like. The machine learning models are trained using training data to form trained machine learning models. The training data is used to modify and fine-tune any weights associated with the machine learning models, as well as record ground truth for where correct answers can be found within the data. As such, the better the training data, the more accurate and effective the machine learning model or technique. According to one embodiment, the machine learning models can be trained using health-related data, which includes patient encounter data, appointment data, patient data, provider schedule data, and the like. The prediction unit 24 thus generates output model data 26 in the form of insights and predictions. The output model data 26 or the stored data 22 can then be conveyed to and ingested by a reporting unit 28. The reporting unit 28 can have stored therein suitable application software for generating one or more user interfaces, dashboards, and reports based on the received data. The dashboards allow the user to prepare selected reports and to visualize the data. By way of example, the reporting unit 28 can employ any suitable visualization software, such as Power BI from Microsoft, Inc., USA. Power BI is a collection of software services, applications, and connectors that work together to convert or transform the input health related data into coherent, visually immersive, and interactive predictions and insights. Those of ordinary skill in the art will readily recognize that the reporting unit 28 and the prediction unit 24 can form part of the same module or unit.

The patient encounter determination unit 20 can also employ the health-related data that is stored in the data model 30 (e.g., model data 36) to determine or calculate selected types of information. For example, the patient encounter determination unit 20 can be employed to process the model data 36 stored in the data model 30 to determine or calculate specific types of information, such as patient encounter data and other types of health-related data. For example, the model data 36 can be processed to determine demand opportunity related data. As used herein, the term “demand opportunity” and associated data is intended to refer to a calculated or measurable impact of recovering or rescheduling appointments, such as lost appointments, that are missed or canceled for manageable reasons, including for example provider centric reasons and/or patient centric reasons. As used herein, the term “manageable reason(s)” is intended to refer to avoidable reasons for cancelling or missing the appointment since the reasons are either under the control of the healthcare provider that can allow for or permit the rescheduling of the appointment, or under the control of the patient, such as a lack of suitable transportation, weather, and the like. As such, the patient encounter determination unit 20 can employ the model data 36 that includes health-related data to classify or categorize the patient encounter data, including lost appointment data, and to quantify the opportunity to recapture the appointment, such as the demand opportunity.

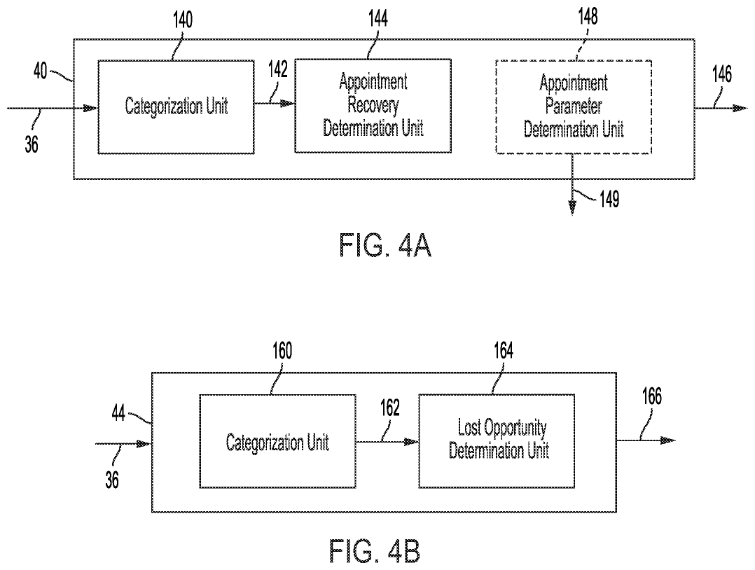

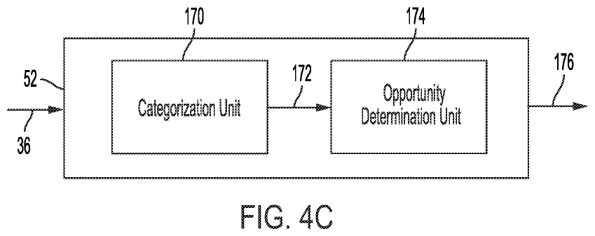

Specifically, as shown in FIGS. 2 and 4A, the patient encounter determination unit 20 can employ an optional demand opportunity determination unit 40 for receiving and processing the model data 36 that includes lost appointment data and schedule data and determining therefrom the number of recoverable lost appointments. For example, the model data 36 can be stored in at least the patient encounter data table 90 and in the provider schedule data table 92, as well as in at least the data tables 102, 104, 106, 116, 122, 126, and 132 of the data model 30. The demand opportunity determination unit 40 can include a categorization unit 140 that is configured to classify or categorize the appointment data in the model data 36 into a series of appointment related categories. The appointment related categories generated by the categorization unit 140 can include a new appointment category indicative of new patient appointments, a completed appointment category indicative of patient appointments that have been completed, and various lost appointment categories, such as a missed appointment category indicative of a patient that misses an appointment (e.g., a patient no-show or the patient left without being seen), an appointments canceled by provider category indicative of appointments that have been canceled by the healthcare provider or by the healthcare facility, and a canceled by patient category indicative of appointments that have been canceled by the patient. Each of the appointment categories can include therein similar appointment related information. As such, an appointment categorized as “canceled” can refer to an appointment that is canceled for selected predetermined or predefined reasons, including illness, weather related reasons, transportation related reasons, and other predefined reasons.

Further, the appointment data in selected ones of the foregoing appointment categories can be further sub-categorized or sub-classified as “canceled—patient recoverable”, which designates or categorizes selected canceled appointment as being canceled according to a selected methodology, such as by auto reminders, through a patient portal, due to family issues, or transportation issues. Appointments of this type are deemed to be recoverable according to a predefined set of rules and logic since they are considered to be manageable reasons for the appointment being lost. The categories employed by the categorization unit 140 can be mapped to related or appropriate sub-categories or classes that can then be used to quantify the recovery opportunity. The canceled type of lost appointments is likely to be recovered by the healthcare facility or healthcare provider by initiating or performing one or more follow-on recovery actions, such as by follow-up and the like. The appointment data in the canceled by provider category can also be subcategorized as “canceled—provider related reasons.” This appointment designation covers the cancellation of the appointments by the provider or healthcare facility for various reasons, such as healthcare provider availability and the like. These various appointment related categories correspond to various types of canceled appointments and can be deemed, in general, to be lost appointments. The categorization unit 140 can then generate classification data 142.

The demand opportunity determination unit 40 can also include an appointment recovery determination unit 144 that receives the classification data 142 generated by the classification unit 140. The classification data 142 can include lost appointment data, such as data associated with missed and canceled appointments. The appointment recovery determination unit 144 can then determine based thereon which of the appointments, such as the lost appointments, in the various categories are potentially recoverable by determining which of the lost appointments were lost for manageable reasons. As used herein, the term “recoverable” or “recoverable appointment” is intended to refer to an assessment based on a set of predefined logic or rules that a previously lost appointment (e.g., missed or canceled appointment) can be recovered, recaptured and/or rescheduled by the healthcare provider or facility by determining whether the appointment was lost for manageable reasons, or whether the appointment slot can be filled with other patients. The factors used to determine the number of recoverable appointments are the defined categories sub-categories and the calculated recovery factors (e.g., percent of appointment recovery) set or defined by each category depending on the specifics or needs of the healthcare provider, historical appointment trends, historical recovery factors, patient behavior trends, and the like. The lost appointment data in the classification data 142 is processed by the appointment recovery determination unit 144 to determine the number, including optionally the percentage, of potentially recoverable appointments from the lost appointment data. The determination of the number of recoverable appointments in the lost appointments data can assist the healthcare facility in determining the amount of revenue that is potentially recoverable. The appointment recovery determination unit 144 can thus generate demand opportunity data (e.g., recoverable appointment data 146).

The demand opportunity determination unit 40 can also employ an optional appointment parameter determination unit 148 for determining, based on the patient encounter data (e.g., lost appointment data) in the classification data 142, one or more appointment parameters 149 associated with the patient appointments. The appointment parameters can include one or more parameters associated with appointments, such as appointment date, appointment time, appointment length or duration, appointment type, patient identification information, healthcare provider information, appointment status (e.g., scheduled, confirmed, canceled, completed, rescheduled, and the like), patient health-related data, patient arrival and check-in information, appointment length information (e.g., preferred length, default length, standard length, optimal length), and the like. More specifically, the appointment parameter determination unit 148 can determine based on the classification data 142 a suitable or optimal length of a patient appointment 149 by employing selected types of input appointment data, including one or more of healthcare provider data, the specialty of the healthcare provider data, historical appointment length data including a minimum and maximum appointment times, as well as optionally employing a default appointment time data. The appointment parameter determination unit 148 can also be optionally configured to determine or to calculate a date or a date range of the patient appointment (or an optimal date or date range) or to determine a status or a type of the appointment. The demand opportunity recovery factors can be determined or can be preset by identifying the desire date range to consider, then setting the appropriate recovery factors by category based on the needs and specifics of the healthcare facility. The factors cab ne adjusted or changed over time or as needed.

The appointment parameter determination unit 148 can also be optionally configured to determine or to calculate a recovery factor or value based on the canceled appointment data portion of the lost appointment data in the classification data 142. As used herein, the term “recovery factor” or “recovery value” is intended to mean a selected portion (includes a percentage) of the number of lost appointments that were canceled or missed for manageable reasons that the appointment parameter determination unit 148 determines can be recovered, rescheduled and converted into subsequent appointments or can be filled with other patient appointments, which in turn can lead to additional revenue for the healthcare facility. The recovery factor helps optimize scheduling and patient utilization. The recovery factor can be a dynamic factor, number or value that can be used by the appointment parameter determination unit 148 to model or drive different behaviors and circumstances specific to each healthcare facility involving appointments. The appointment parameter determination unit 148 uses the total number of lost appointments that were lost for manageable reason and applies the determined or calculated recovery factor to each category of lost appointments, such to a missed appointments category and to a canceled appointment category, so as to determine the number of lost appointments that can be recovered.

The appointment parameter determination unit 148 can be further configured to apply a prestored reimbursement rate dynamic factor to the recoverable lost appointments to determine or to calculate a potential reimbursement amount based on the number of lost appointments that were lost for manageable reasons. The reimbursement rate dynamic factor can refer to the variable factors that can change over time and influence the rate or amount of reimbursement or payment that healthcare providers receive for their services. The factor is often used to adjust reimbursement rates based on various criteria or conditions. The reimbursement rates are typically determined through negotiations between the healthcare providers (such as hospitals and physicians) and payers (such as insurance companies or government programs like Medicare and Medicaid). The rates can be influenced by a variety of factors, and the dynamic factor is one of them.

By simple way of example, the categorization unit 140 of the demand opportunity determination unit 20 categorizes or classifies the lost appointment data and generates classification data 142, which includes the categorized lost appointment data. The appointment recovery determination unit 144 can then determine based thereon which of the lost appointments in the various categories are potentially recoverable since the appointments were lost for manageable reasons. The appointment parameter determination unit 148 can then employ the appointment volume associated with the manageable (or recoverable) cancelation reasons, either in total or per category, and applies the recovery factor to determine the number or percentage of lost appointments that can be recovered. The appointment parameter determination unit 148 can then apply the reimbursement rate dynamic factor to the recoverable appointments (e.g., appointments that were determined to be lost for manageable reasons) to determine a revenue amount expected to be obtained by rescheduling the recoverable appointments. The demand opportunity determination unit 40 can thus generate the recoverable appointment data 146 and/or the appointment parameter data 149 that can be processed by the prediction unit 24 or can be directly conveyed to the reporting unit 28. The prediction unit 24 can employ one or more machine learning models to the recoverable appointment data 146 and/or the appointment parameter data 149 to extract or determine insights or predictions based on the data.

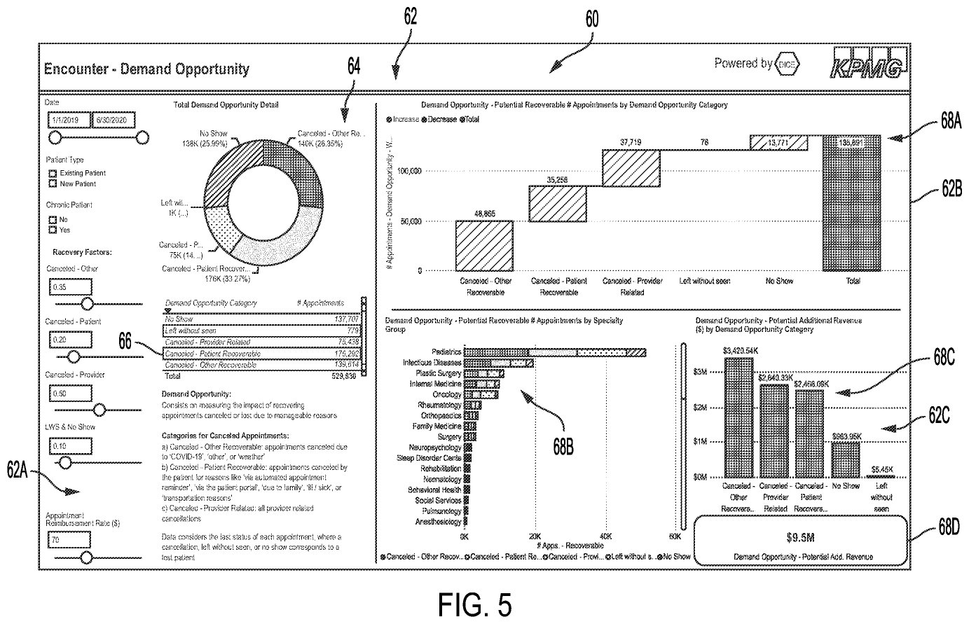

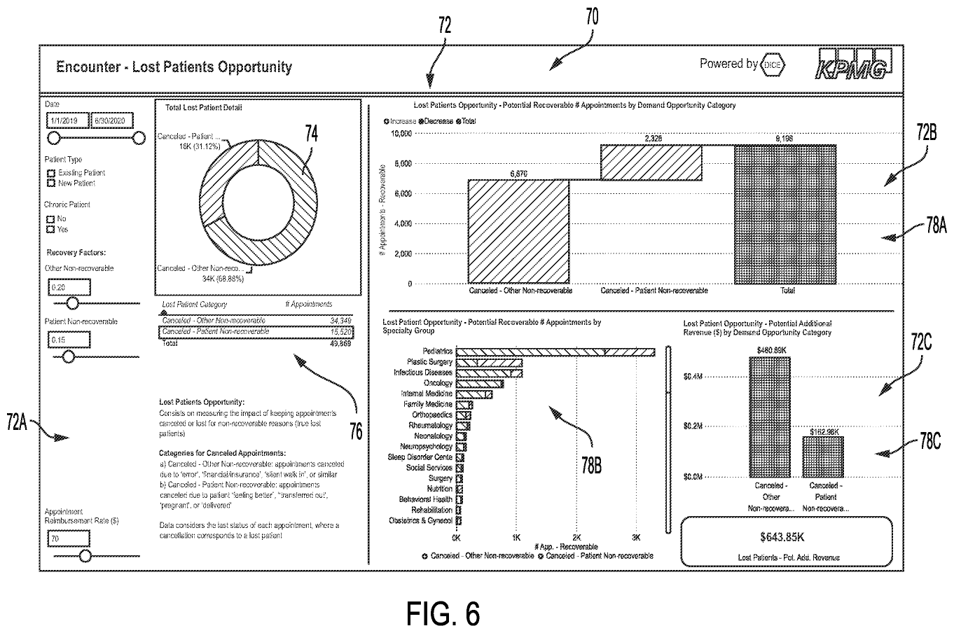

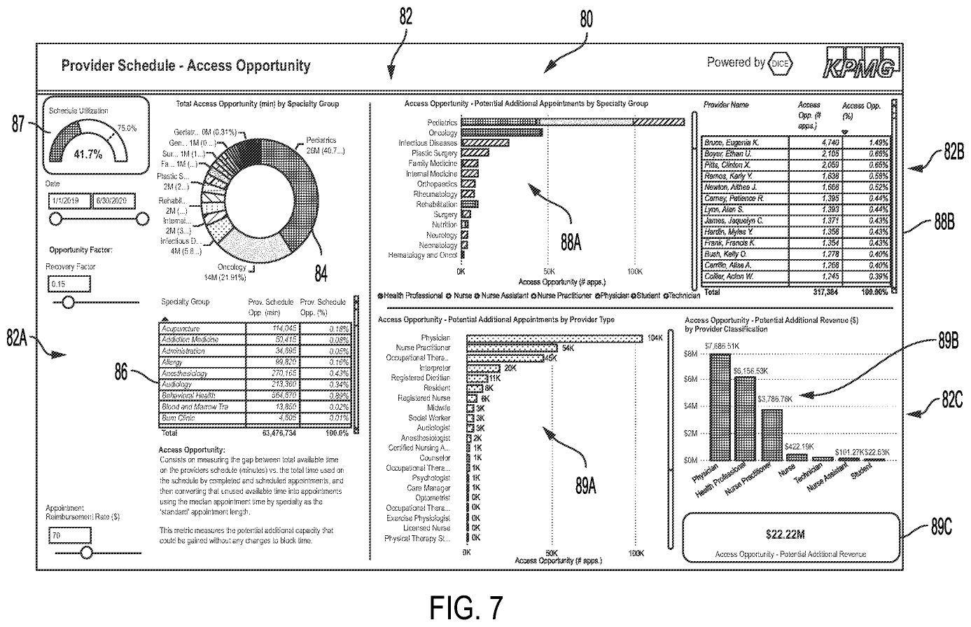

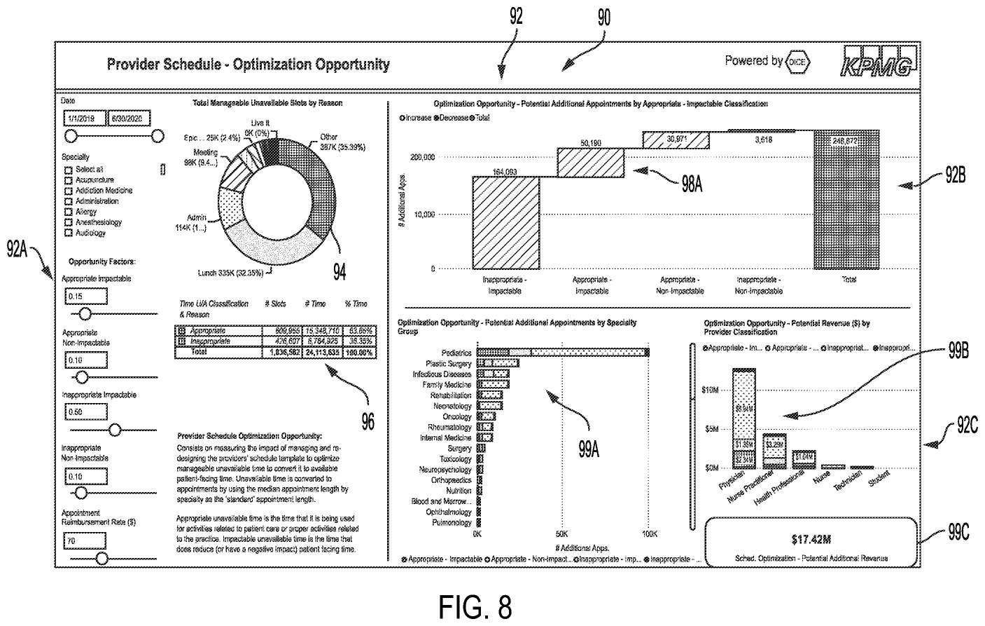

The reporting unit 28 of the present invention can receive and process the prediction data 26 and can generate, either through a user interface generator or via prepackaged visualization software, one or more user interfaces for displaying selected data, such as the prediction data 26 and/or the stored health data 22. The user interface examples shown in FIGS. 5-8 process the input health data and display information that allows the user to identify and measure the additional revenue in dollars on core patient access and healthcare provider schedule aspects of the healthcare facility, such as patient demand opportunity (FIG. 5), lost patients opportunity (FIG. 6), patient access opportunity (FIG. 7), and optimization opportunities (FIG. 8).

For example, the reporting unit 28 can generate for display on a suitable display element the user interface shown in FIG. 5. When the user of the system 10 wishes to view patient demand opportunity related information associated with the healthcare facility, the reporting unit 28 can process the demand opportunity data (e.g., recoverable appointment data 146 and/or appointment parameter data 149) generated by the demand opportunity determination unit 40 or the prediction data 26 and can generate the demand opportunity user interface 60. The illustrated user interface 60 can include the window 62 (herein generally referred to as a window, a frame, or a page) that displays selected information in selected regions or panes thereof. The graphical user interface can be a series or system of interactive visual components that can be executed by suitable software, such as that implemented by the reporting unit 28. The user interface can hence include windows, pane elements, tab elements, sub-tab elements, ribbons, bars, soft buttons, icons, objects, menus, and graphical elements that convey information, execute commands, and represent actions that can be taken by the user. The objects or icons can remain static or can change or vary when the user interacts with them. Hence, the reporting unit 28 can include suitable application software that can generate the user interfaces of the present invention. Those of ordinary skill in the art will readily recognize, based on the teachings herein, that the window 62 can display any selected type of information associated with patient demand opportunity and can arrange the information in any selected manner. According to one embodiment, the window 62 includes one or more frames or pane elements, including the pane element 62A that is disposed along the left-hand side of the window 62 and the vertically stacked pane elements 62B and 62C that are disposed along the right-hand side of the window 62. The pane element 62A can be configured to display details associated with the total demand opportunity associated with the healthcare facility. The demand opportunity data can be displayed in any selected format. For example, the pane element 62A can include a graphical element, such as a circle chart 64, that displays selected types of appointment information, such as a breakdown of the total lost appointments that were either canceled, missed for patient no-shows, missed since the patient left without being seen, and patient canceled appointments, as well as the total number of appointments that the demand opportunity determination unit 40 determines are potentially recoverable. Specifically, the lost patient appointment information can include information associated with the status or category of the lost appointments. The lost appointments can be categorized by the demand opportunity determination unit 40 (e.g., the categorization unit 140) in selected ways, as noted above, such as, for example, as various types of canceled or missed appointment categories, including a no-show appointment category, a canceled appointment category, a left without being seen appointment category, and the like. The information displayed in the circle chart 64 can also be presented in tabular format in table 66. The pane element 62A can also display date information in a selected range of date formats, patient type (e.g., new or existing patient), patient health status indicator (e.g., chronic or non-chronic), patient reimbursement rate information, demand opportunity information, patient appointment related information, amount of lost or recoverable revenue, and the like.

The illustrated pane element 62B disposed along the upper right-hand side of the window 62 can include a graphical element, such as a line chart 68A, that sets forth the lost appointment information and the potential recoverable appointments per category of lost appointments. For example, the line chart 68A can display the assessment of the appointment recovery determination unit 144 regarding the number of recoverable appointments per displayed category, as a well as a total number of recoverable appointments. The pane element 62C located on the lower right-hand side of the window 62 displays another graphical element, such as the line chart 68B, that displays the potentially recoverable appointments by specialty practice (e.g., type of healthcare provider) in the healthcare facility. The user interface 60 can also display the information associated with the lost appointments by categorizing the appointments by provider specialty or practice, by healthcare facility, by facility location, by day or time, and the like. The pane element 62C also includes a graphical element, such as line chart 68C, that displays the potential additional revenue that the healthcare facility or provider can recoup based on the recovery of a selected number or percentage of appointments in the different categories of lost appointments. The appointment parameter determination unit 148 can determine the revenue based on the determined recovery factor, which can be displayed as a total revenue number 68D.

The patient encounter determination unit 20 can also optionally include a lost patients opportunity determination unit 44 for receiving and processing the output model data 36 and for determining a lost patients opportunity related to lost appointments that are lost for non-recoverable reasons. As used herein, the term “lost patient opportunity” and “lost opportunity” and associated data refers to the ability to determine, calculate or measure a financial and/or resource impact of keeping, maintaining, or replacing the patient appointments that are lost for non-recoverable reasons. As used herein, the term “non-recoverable” or “non-recoverable reason” is intended to refer to the reasons for missing or canceling appointments that are under the control of the patient and are triggered by some specific provider behavior or operational constraint. For example, the behavior or constraints can include excessive appointment lag (e.g., days the patient needs to wait to see a doctor) leading to the patient cancelling the appointment, or long wait times after checking in for the appointment leading to a missed appointment (e.g., patient leaving), among other reasons. The reasons can also include scheduling or billing errors, insurance issues, and the like. The lost patients opportunity determination unit 44 can optionally include a categorization unit 160 for categorizing the model data 36, which includes lost appointment data, into a series of categories. According to one embodiment, the lost appointments can be categorized into two or more different appointment related categories, including, for example, into a first category that includes non-recoverable canceled appointments, which includes appointments that are canceled due to provider centric reasons such as appointment or scheduling errors, insurance or billing issues, and the like and are hence deemed to be non-recoverable. The categorization unit 160 can also employ a second category that includes patient non-recoverable canceled appointments that includes appointments that are canceled by the patient due to, for example, a change in health status of the patient (e.g., the patient is feeling better), transfer of the patient to a location outside of the healthcare facility, and the like. The foregoing categories are related to canceled appointments that are not recoverable. The categorization unit 160 can generate categorization data 162 indicative of the various appointment categories. The categorization data 162 is then received and processed by a lost opportunity determination unit 164 for determining, based on the categorization data 162, which includes lost appointment data, the lost opportunity associated with the patient appointments that are lost for non-recoverable reasons. The estimated lost revenue can be determined, according to one embodiment, in a manner similar to the calculation or determination of the demand opportunity. For example, the lost patients opportunity determination unit 44 can classify or categorize the cancelation reasons based on non-recoverable reasons using the categorization unit 160 and then can determine the volume of appointments per category. Then, using a recovery factor for each of the cancellation reasons, the lost patients opportunity determination unit 44 employing the lost opportunity determination unit 164 can determine (e.g., quantify) the number of appointments that can be potentially recovered from the appointments lost for non-recoverable reasons. The recovery factor is the percentage or number of lost appointments that were canceled or missed for manageable reasons that can be rescheduled and converted into subsequent appointments or can be filled with other patient appointments. The patient access determination system 10 can then determine or calculate, using the reimbursement rate, the additional revenue that can be generated from all the recovered or recaptured appointments. The lost opportunity determination unit 164 can generate lost opportunity output data 166. The prediction unit 24 can employ one or more machine learning models to process the lost opportunity output data 166 to extract or to determine insights or predictions based on the data.

When the user of the system 10 wishes to view lost appointments or lost patient related information associated with the healthcare facility, the reporting unit 28 can ingest the lost opportunity output data 166, functioning as the stored health data 22, or employ the prediction data 26 and generate the user interface 70, as shown for example in FIG. 6. The illustrated user interface 70 can include the window 72 that displays selected information in selected regions or panes thereof. Those of ordinary skill in the art will readily recognize, based on the teachings herein, that the window 72 can display any selected type of information associated with the lost appointment data and the lost opportunity data and can arrange the information in any selected manner. According to one embodiment, the window 72 displays total lost patient data based on one or more selected appointment categories including canceled appointments, lost opportunity data, lost patient opportunity by healthcare provider specialty, and a determination of an estimated lost revenue (e.g., lost patient opportunity) based on one or more of the lost appointment data and the lost patient data. The window 72 can also display information associated with a determination by the lost patients opportunity determination unit 44 of the number of appointments that are recoverable from the canceled appointments as well as the potential revenue generated by the recovery of the selected appointments.

According to one embodiment, the window 72 includes one or more frames or pane elements, including the pane element 72A that is disposed along the left-hand side of the window 72 and the vertically stacked pane elements 72B and 72C that are disposed along the right-hand side of the window 72. The pane element 72A can be configured to display details associated with the total lost patient opportunity associated with the healthcare facility. The lost patient opportunity data can be displayed in any selected format. For example, the pane element 72A can include a graphical element, such as a circle chart 74, that displays selected types of appointment information, such as a breakdown of the total lost appointments that correspond to appointments canceled for non-recoverable reasons and appointment canceled by patient for non-recoverable reasons. Specifically, the lost patient appointment information can include information associated with the status or category of the lost appointments. The lost appointments (e.g., canceled appointments) can be categorized by the lost patients opportunity determination unit 44 in selected ways, as noted above, such as for example as various types of canceled appointment categories, including a canceled by patient for non-recoverable reasons and canceled for other non-recoverable reasons. The information displayed in the circle chart 74 can also be presented in tabular format in table 76. The pane element 72A can also display date information in a selected range of date formats, patient type (e.g., new or existing patient), patient health status indicator (e.g., chronic or non-chronic), recovery factor information, patient reimbursement rate information, and the like.

The illustrated pane element 72B located along the upper right-hand side of the window 72 can include a graphical element, such as a line chart 78A, that sets forth lost appointment and potential recoverable appointment information based on the lost appointment categories set forth in pane 72A. The lower right-hand side pane element 72C displays a graphical element (e.g., line chart 78B) that displays the lost patient opportunity and potentially recoverable appointments by specialty practice or group (e.g., healthcare providers) in the healthcare facility. The pane element 72C can also display the potential additional revenue associated with the lost patient opportunity information. For example, the revenue associated with each category set forth in the other pane elements can be determined by the lost patients opportunity determination unit 44 and displayed via a line chart 78C that displays the potential additional revenue that the healthcare facility or provider can recoup based on the recapture of a selected percentage or number of appointments in the different categories of lost appointments. The revenue total can be displayed.

The patient encounter determination unit 20 can further include an optional access opportunity determination unit 48 for receiving and processing the model data 36 and for determining the total demand or access opportunity that the patient has to appointments available to them at a selected healthcare facility. As used herein, the term “access opportunity” or “patient access opportunity” refers to the ability to determine, calculate or measure the difference or gap between the total available time for the healthcare facility to schedule or complete patient appointments and the amount or number of actual time scheduled for patient appointments scheduled or completed patient appointments with the healthcare facility. For example, the healthcare facility can determine the total number or time associated with potentially available patient appointments across some or all of the practice areas by accessing and processing possible patient appointment scheduling data for the practice groups. This information can form part of the input health data. The access opportunity determination unit 48 can receive, process or determine the number of actual scheduled patient appointments across the practice groups from this data. Specifically, the access opportunity determination unit 48 determines the total time gap between available appointment time and actual schedule appointment time, and then can convert the unused or non-scheduled appointment time available in the schedule of the practice groups into actual patient appointments using selected types of data, including for example, a selected appointment time length (e.g., a median appointment time length) by specialty as a standard or default appointment time length. The access opportunity determination unit 48 can then optionally apply a reimbursement rate to the available additional appointments to determine potential revenue associated with the additional available appointments. The prediction unit 24 can employ one or more machine learning models to process access opportunity data to determine insights or predictions based on the data.

When the user of the system 10 wishes to view or display data associated with an access opportunity of a healthcare provider's schedule (e.g., potential available appointments) associated with the healthcare facility, the reporting unit 28 can generate, based on the access opportunity data generated by the access opportunity determination unit 48 or based on the associated prediction data 26, the user interface 80, as shown for example in FIG. 7. The illustrated user interface 80 can include the window 82 that displays selected information in selected regions or panes thereof. Those of ordinary skill in the art will readily recognize, based on the teachings herein, that the window 82 can display any selected type of information associated with the access opportunity of a patient to a healthcare facility. According to one embodiment, the window 82 displays schedule utilization data (e.g., access opportunity data) associated with the amount of schedule or appointment capacity currently fulfilled by patient appointments, total patient access opportunity based on healthcare provider specialty, access opportunity data, and potential additional revenue data the healthcare facility can potentially raise based on the access opportunity.

According to one embodiment, the window 82 includes one or more frames or pane elements, including the pane element 82A that is disposed along the left-hand side of the window 82 and extends from top to bottom and the vertically stacked pane elements 82B and 82C that are disposed along the right-hand side of the window 82. The pane element 82A can be configured to display details associated with the patient access opportunity (e.g., schedule utilization) associated with a healthcare facility. Specifically, the access opportunity can correspond to the difference or gap between the total amount of time, in any selected time increment, such as minutes, that are available in the providers schedule to accommodate patient appointments and the actual amount time associated with scheduled and completed appointments. The access opportunity determination unit 48 provides this information such that the healthcare facility can attempt to convert the unused available appointment time into actual patient appointments using a median appointment time by specialty as a standard appointment time length. As such, the access opportunity determination unit 48 can determine the potential additional appointment capacity of the healthcare facility or specialty group based on existing available appointment time.

The illustrated pane element 82A can include a graphical element, such as a circle chart 84, that displays the various specialty groups associated with or comprising the healthcare facility that have time available to schedule patient appointments and the percentage of open time. The information displayed in the circle chart 84 can also be presented in tabular format in table 86. The pane element 82A can also display date information in a selected range of date formats, recovery factor information, patient reimbursement rate information, and the like. The pane element 82A can also employ a graphical element 87 that displays by a visual indicator the total appointment time based on percentage (e.g., 100%) and the current total utilized capacity. The pane element 82B located along the upper right-hand side of the window 72 can include a line chart 88A that displays the access opportunity (e.g., available appointment time) per practice group. The access opportunity can be displayed by unused or available time or by revenue. The pane element 82B located along the upper right-hand side of the window 82 can also display in a table 88B the access opportunity per specialty group, provider, or individual healthcare personnel.

The lower right-hand side pane element 82C displays the line chart 89A that displays the access opportunity associated with each specialty group or provider type. Specifically, the line chart 89A shows the potential additional appointments by provider type that can be recovered or recaptured by the healthcare facility. The pane element 82C also displays a line chart 89B that displays the potential additional revenue that can be recovered or recaptured by enhanced or increased appointments by provider classification. The pane element 82C can also include a graphical element 89C that shows the total revenue that can be recovered based on enhanced appointment utilization. The total revenue can be determined by initially identifying the available and unused appointment or time slots per specialty and per provider type, and then using the median appointment length by specialty. The system can calculate the number of available slots per specialty and can then multiply those identified slots by the appointment reimbursement rate factor (healthcare provider dependent) to determine the total opportunity 89C. The total opportunity or revenue is then segmented by provider type.

The demand opportunity determination unit 40 can further include an optional optimization opportunity determination unit 52 for receiving and processing the model data 36 and for determining an optimization opportunity associated with managing or optimizing the unavailable appointment time of the healthcare provider and/or the healthcare facility. As used herein, the term “optimization opportunity” or “provider schedule optimization opportunity” refers to the ability to determine, calculate or measure a financial or resource impact on the healthcare facility of managing and/or optimizing unavailable appointment time. The system of the present invention can identify (e.g., mark or flag) the appointments that are deemed unavailable for non-valid reasons (e.g., other). These appointments can be recovered by, for example, modifying the provider and specialty schedule and through provider education. The unavailable flag can then be removed, and the appointment time or slot can be deemed to be available for patient appointments, The potential revenue associated with the recovered appointment time can then be determined. The unavailable appointment time corresponds to the total appointment time that is unavailable for patient appointments. The specifics of the optimization opportunity determination unit 52 is shown in FIG. 4C. The illustrated optimization opportunity determination unit 52 can include a categorization unit 170 that can be configured to receive the model data 36, which includes appointment data, and categorize an unavailable appointment time portion of the appointment data into multiple different appointment related categories. The categories can include, for example, an appropriate unavailable appointment time category and an inappropriate unavailable appointment time category. The appropriate unavailable appointment time corresponds to appointment time that is being used by personnel of the healthcare facility for appropriate activities, such as activities related to patient care or other patient centric activities associated with the healthcare facility. The appropriate unavailable appointment time can be further sub-categorized into impactable appropriate unavailable appointment time and non-impactable appropriate unavailable appointment time. The impactable appropriate unavailable appointment time corresponds to activities that reduces the total appointment time available to patients but can be addressed or reduced (e.g., impacted). The activities associated with this subcategory can include managing and adjusting appointment schedules in real time, considering factors such as appointment cancellations, appointment rescheduling, or urgent care needs. This type of appointment time is considered as potentially recoverable appointment time. The non-impactable appropriate unavailable time is the appointment time that has been reserved, blocked, or held for any reason that is part of the normal or typical operation of the healthcare provider or facility (e.g., meetings, hospital rounds, surgery, and the like) and typically cannot be changed (e.g., non-impactable). This type of appointment time is not considered as recoverable appointment time. The inappropriate unavailable appointment time, where almost every appointment time can be classified as impactable, is the appointment time that has been reserved, blocked or held for any reason that is not part of the normal operations of the healthcare provider. This type of unavailable appointment time can be considered as potentially recoverable appointment time. The foregoing types of unavailable appointment times are categorized by the categorization unit 170. The categorization unit 170 can then generate categorization data 172 that is indicative of the various categories of unavailable appointment time.

The optimization opportunity determination unit 52 can also include an opportunity determination unit 174 for determining the portion of the impactable unavailable appointment time that can be converted into available appointment time. The opportunity determination unit 174 hence is able to differentiate and quantify the correct number or amount of unavailable appointment time that can be allotted back or converted into available appointment time. The unavailable appointment time can be converted into available appointment time by, for example, identifying or quantifying the impactable portion of the unavailable appointment time that is recapturable by identifying the correct type of appointment activity or the reasons that the appointment time is being held. The process is similar to the process used for unavailable time. This process uses the impactable/non-impactable and appropriate/inappropriate categories or classifications to segment the opportunity and then determine how much time or how many appointments can be recovered. Once the total recoverable appointment time is identified from the unavailable appointment time, the opportunity determination unit 174 then applies a preselected appointment time length (e.g., a median appointment length) by provider specialty as a standard appointment length to determine the total number of potential recoverable appointments are available from the total recoverable appointment time. Specifically, once the total time per unavailable or impactable reason is determined or calculated and converted into a number of potential appointments (e.g., by using the median appointment length by provider specialty), the opportunity determination unit 174 can apply one or more recovery factors to determine or to calculate the actual number of appointments that can be recovered. Once the number of recoverable appointments is calculated, the opportunity determination unit 174 can apply a reimbursement rate to convert the number of appointments to potential revenue (e.g., in dollars). The opportunity determination unit 174 can generate appointment opportunity data 176, which can form part of the stored health data 22. The prediction unit 24 can employ one or more machine learning models to optimization opportunity data generated by the optimization opportunity determination unit 52 to extract or determine insights or predictions based thereon.

When the user of the patient access determination system 10 wishes to view data associated with an optimization opportunity of a provider's schedule associated with a healthcare facility, the reporting unit 28 can receive the stored data 22 or the prediction data 26 and generate based thereon the user interface 90, as shown for example in FIG. 8. The illustrated user interface 90 can include the window 92 that displays selected information in selected regions or panes thereof. Those of ordinary skill in the art will readily recognize, based on the teachings herein, that the window 92 can display any selected type of information associated with the optimization opportunity. According to one embodiment, the window 92 displays various category information associated with appropriate and available appointments (e.g., time slots), optimization opportunities by specialty practice, and potential revenue data associated with optimization opportunities.

According to one embodiment, the window 92 includes one or more frames or pane elements, including the pane element 92A that is disposed along the left-hand side of the window 92 and extends from top to bottom and the vertically stacked pane elements 92B and 92C that are disposed along the right-hand side of the window 92. The pane element 92A can be configured to display details associated with the optimization opportunity associated with the healthcare facility. Specifically, the optimization opportunity can correspond to the ability to manage and optimize unavailable appointment time and converting the unavailable appointment time into available appointment time. Specifically, the optimization opportunity determination unit 52 can determine the unavailable appointment time and then convert the unavailable appointment time into available appointment time (e.g., number of available appointment times) by using a preselected appointment time length (e.g., median appointment time). The pane element 92A can include a circle chart 94 that displays the various types of manageable unavailable appointments and the reasons associated with the unavailable appointments. The unavailable appointment time information displayed in the circle chart 94 can also be presented in tabular format in table 96. For example, the table 96 can show the unavailable appointment time data segmented into appropriate unavailable appointment time data and inappropriate unavailable appointment time data. The pane element 92A can also display date information in a selected range of date formats, specialty provider information, and opportunity factor information, patient reimbursement rate information, and the like. The pane element 92B located along the upper right-hand side of the window 92 can include a line chart 98A that displays the unavailable appointment time information by type, such as inappropriate impactable unavailable appointment time, appropriate impactable unavailable appointment time, appropriate non-impactable unavailable appointment time, inappropriate non-impactable unavailable appointment time, and a total unavailable appointment time.

The lower right-hand side pane element 92C displays the line chart 99A that displays the potential additional appointments (e.g., optimization opportunity) determined by the opportunity determination unit 174 segmented by specialty group or provider type. Specifically, the line chart 99A shows the potential recoverable unavailable appointments by provider type that can be recovered or recaptured by the healthcare facility. The pane element 92C also displays a line chart 99B that displays the potential additional revenue that can be generated by recovering the identified unavailable appointment times, segmented by type of unavailable appointment time. The pane element 92C can also include a graphical element 99C that shows the total revenue that can be recovered based on enhanced appointment recapture. After identifying the unused time slots that are flagged as unavailable per specialty and per provider type, and using the median appointment length by specialty practice, the system can calculate the number of available slots per specialty and multiply those slots by the appointment reimbursement rate factor to determine the total opportunity or revenue 92C. The total opportunity is then segmented by provider type.

The patient access determination system 10 of the present invention thus has the ability to allow the user to model behavior and measure revenue opportunity with the use of user input dynamic parameters (e.g., opportunity factors, recovery factors, and reimbursement rates), and by calculating the opportunity across the healthcare facility. For example, the user interface 90 displays user selectable opportunity factors and reimbursement rates that can be employed with the optimization opportunity.

The patient access determination system 10 of the present invention can also automatically provide suggested steps to modify the provider schedule in such a way so as to optimize the provider's hours to accommodate as many patients as possible. Further, the patient access determination system 10 can automatically generate a patient behavior list providing visibility of the probability of a patient to attend, reschedule, cancel, or no-show a scheduled appointment. The list can provide better insight into patient behavior that can be used to improve scheduling practices of the healthcare facility.

The patient access determination system 10 can further generate information, such as an index/ratio, that measures the appointment leakage and appointment volume and provide a clear and easy way to identify the specialties/providers that require additional support and training on managing appointments and properly scheduling appointments. To that end, the patient access determination system 10 can identity bottlenecks in the referral process and provide a list of steps to resolve the bottlenecks and reduce the time from referral order to patient encounter.

The patient access determination system 10 of the present invention also helps to identify, measure, and quantify overall patient access to a healthcare facility, a provider schedule, and referral opportunities in a single and integrated system, thus providing the user with the ability to clearly identify areas of economic opportunity considering all aspects of the operation of the healthcare facility. The system also helps the healthcare facility to reduce the amount of wasted or unused time for seeing patients. The system allows the user to quantify and to consolidate, in revenue dollars, patient access, provider schedule, and referrals that the organization can achieve if proper optimization policies and procedures are implemented.

The patient encounter determination unit 20 of the patient access determination system 10 can also be configured to provide a year-over-year (YoY) analysis on selected types of appointment data, including for example on completed appointments, canceled appointments, appointments where the patient did not show up, appointments where the patient left without being seen by a healthcare provider, rescheduled appointments, and any other final status appointments to understand the behavior of the healthcare facility over time. The patient encounter determination unit 20 can also determine or calculate an index, based on the differential month over month appointment data, that defines if the overall appointment trend is growing (e.g., a positive value), declining (e.g., a negative value), or remains neutral. The larger the absolute value of the index value for each of the metrics represents if the change is linear or exponential. The index is the gap between one month and the previous month (e.g.: February-January, March-February, etc.). The index allows the user to see if the increase or decrease of appointments is flat (close to 0), steady and consistent change (like a 45-degree angle) or exponential change (rate will be a curve).

The reporting unit 28 can also be optionally configured to generate a user interface that displays schedule time by appointment classification. The schedule time can be segmented or categorized and displayed as being filled or unfilled, available or unavailable, and if the time can be considered for an optimization opportunity (e.g., inappropriate and impactable) or not (e.g., appropriate and non-impactable). The displayed appointment data can support the provider schedule redesign process and allows the user to identify where the biggest optimization opportunity exists.

The reporting unit 28 of the patient access determination system 10 can be configured to optionally generate a user interface that displays for comparison provider appointment utilization data with administrative performance metrics used to administer healthcare providers. The patient encounter determination unit 20 can dynamically calculate a clinical Full Time Employee (cFTE) rate for each of the healthcare providers, and then calculate weekly performance metrics and the cFTE ratio. These values, along with a visualization of the information that compares the total available time for the provider vs. the cFTE ratio allows the user to identify the impact of the available appointment time (result from scheduling practices) on the overall cFTE. The displayed data can be used to identify areas of opportunity and the providers that need operational optimization.

Further, in order to support the scheduling decision making process, the patient access determination system can perform a basic analysis to determine or forecast if a patient is likely to complete an appointment. The appointment analysis is based on patient historical data across all the different specialties and providers contained in the data. The analysis shows the probability for the patient to attend an appointment based on the appointment lag for all specialties and a specific analysis by specialty by time period (e.g., year). The prediction unit 24 can be employed to enhance this analysis using selected information, such as environmental signals (e.g., weather, traffic, seasonality, etc.) as additional factors potentially affecting or impacting the patient capacity to complete, cancel, or no-show for a scheduled appointment.

### Exemplary Hardware

It is to be understood that although the invention has been described above in terms of particular embodiments, the foregoing embodiments are provided as illustrative only and do not limit or define the scope of the invention. Various other embodiments, including but not limited to those described herein, are also within the scope of the claims. For example, elements, units, modules, tools, engines, and components described herein may be further divided into additional components or units or joined together to form fewer components or units for performing the same function.

Any of the functions disclosed herein may be implemented using means for performing those functions. Such means include, but are not limited to, any of the components or units disclosed herein, such as the electronic or computing device components described herein.

The techniques described above and below may be implemented, for example, in hardware, one or more computer programs tangibly stored on one or more computer-readable media, firmware, or any combination thereof. The techniques described above may be implemented in one or more computer programs executing on (or executable by) a programmable computer or electronic or computing device having any combination of any number of the following: a processor, a storage medium readable and/or writable by the processor (including, for example, volatile and non-volatile memory and/or storage elements and non-transitory mediums), an input device, an output device, a display, and the like. Program code may be applied to input entered using the input device to perform the functions described herein and to generate output using the output device.

The term computer, computing system, computer system, computing device, or electronic device, as used herein, can refer to any device that includes a processor and optionally a computer-readable memory capable of storing computer-readable instructions, and in which the processor can execute the computer-readable instructions in the memory. The terms computer system and computing system refer herein to a system containing one or more computing devices.

Embodiments of the patient access determination system 10 of the present invention include features which are only possible and/or feasible to implement with the use of one or more electronic devices, processors, and/or other elements of a computer system. Such features are either impossible or highly impractical to implement mentally and/or manually. For example, embodiments of the present invention may operate on digital electronic processes which can only be created, stored, modified, processed, and transmitted by computing devices and other electronic devices. Such embodiments, therefore, address problems which are inherently computer-related and solve such problems using computer technology in ways which cannot be solved manually or mentally by humans.

Any claims herein which affirmatively require an electronic device, a computing device, a processor, a memory, storage, or similar computer-related elements, are intended to require such elements, and should not be interpreted as if such elements are not present in or required by such claims. Such claims are not intended, and should not be interpreted, to cover methods and/or systems which lack the recited computer-related elements. For example, any system or method claims herein which recite that the claimed method is performed by a computer, a processor, a memory, a storage device or element, and/or similar computer-related element, is intended to, and should only be interpreted to, encompass systems and methods which are performed by the recited computer-related element(s). Such a system and method claim should not be interpreted, for example, to encompass a system or method that is performed mentally or by hand (e.g., using pencil and paper). Similarly, any product or computer readable medium claim herein which recites that the claimed product includes a computer, an electronic device, a processor, a memory, a storage element, and/or similar computer-related element, is intended to, and should only be interpreted to, encompass products which include the recited computer-related element(s) or are executable on the recited or related elements. Such a product claim should not be interpreted, for example, to encompass a product that does not include the recited computer-related element(s) or are not able to be executed on the recited element.

Embodiments of the present invention solve one or more problems that are inherently rooted in computer technology. There is no analog to this problem in the non-computer environment, nor is there an analog to the solutions disclosed herein in the non-computer environment.

Furthermore, embodiments of the present invention represent improvements to computer and communication technology itself. For example, the patient access determination system 10 of the present can optionally employ one or more specially programmed or special purpose computers in an improved computer system, which may, for example, be implemented within a single computing device. Further, the patient access determination system 10 of the present invention can be configured or implemented using one or more computing or electronic devices as described herein, such as a server, a computer, a processor, a memory, storage, and the like.

Each computer program within the scope of the claims may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random-access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; CD-ROMs; solid state device; and the like. Any of the foregoing may be supplemented by, or incorporated in, specially designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk or a separate disk. These elements can also be found in a conventional desktop or workstation computer or server as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

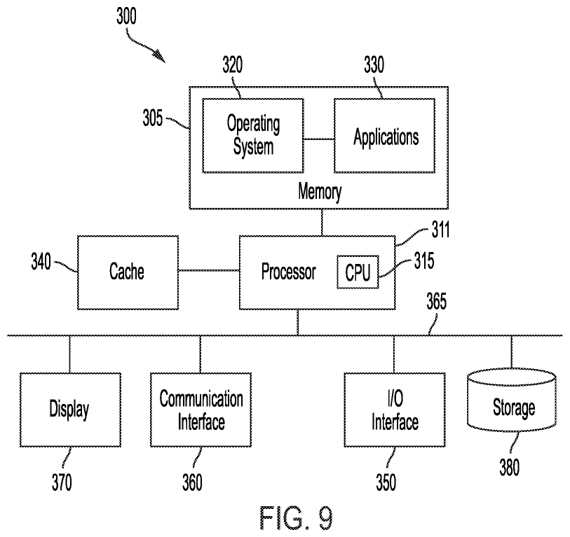

It should be appreciated that various concepts, systems, and methods described above can be implemented in any number of ways, as the disclosed concepts are not limited to any particular manner of implementation or system configuration. Examples of specific implementations and applications are discussed below and shown in FIG. 9 primarily for illustrative purposes and for providing or describing the operating environment of the system of the present invention. The system 10 and/or elements or units or engines thereof can employ one or more electronic or computing devices, such as one or more servers, clients, computers, laptops, smartphones and the like, that are networked together, or which are arranged so as to effectively communicate with each other. The network can be any type or form of network. The devices can be on the same network or on different networks. In some embodiments, the network system may include multiple, logically grouped servers. In one of these embodiments, the logical group of servers may be referred to as a server farm or a machine farm. In another of these embodiments, the servers may be geographically dispersed. The electronic devices can communicate through wired connections or through wireless connections. The clients can also be generally referred to as local machines, clients, client nodes, client machines, client computers, client devices, endpoints, or endpoint nodes. The servers can also be referred to herein as servers, server nodes, or remote machines. In some embodiments, a client has the capacity to function as both a client or client node seeking access to resources provided by a server or server node and as a server providing access to hosted resources for other clients. The clients can be any suitable electronic or computing device, including for example, a computer, a server, a smartphone, a smart electronic pad, a portable computer, and the like, such as the illustrated electronic or computing device 300. The system 10 or any associated units or components of the system 10 can employ one or more of the illustrated computing devices and can form a computing system. Further, the server may be a file server, application server, web server, proxy server, appliance, network appliance, gateway, gateway server, virtualization server, deployment server, SSL VPN server, or firewall, or any other suitable electronic or computing device, such as the electronic device 300. In one embodiment, the server may be referred to as a remote machine or a node. In another embodiment, a plurality of nodes may be in the path between any two communicating servers or clients. The data access and risk management system 10, which includes the query control unit 12, the machine learning model unit 18, the mapping and conversion unit 22, the risk mitigation engine 80, the access verification unit 24, the analyzer unit 130, and the bad actor detection unit 150, can be stored on or implemented by one or more of the electronic or computing devices described herein (e.g., clients or servers), and the hardware associated with the electronic devices, such as the processor or CPU and memory or storage described below and herein.

FIG. 9 is a high-level block diagram of an electronic or computing device 300 that can be used with the embodiments disclosed herein. Without limitation, the hardware, software, and techniques described herein can be implemented in digital electronic circuitry or in computer hardware that executes firmware, software, or combinations thereof. The implementation can include a computer program product (e.g., a non-transitory computer program tangibly embodied in a machine-readable storage device, for execution by, or to control the operation of, one or more data processing apparatuses, such as a programmable processor, one or more computers, one or more servers and the like).

The illustrated electronic device 300 can be any suitable electronic circuitry that includes a main memory unit 305 that is connected to a processor 311 having a CPU 315 and a cache unit 340 configured to store copies of the data from the most frequently used main memory 305. The electronic device can implement the process flow identification system 10 or one or more elements of the process flow identification system.

Further, the methods and procedures for carrying out the methods disclosed herein can be performed by one or more programmable processors executing a computer program to perform functions of the invention by operating on input data and generating output. Further, the methods and procedures disclosed herein can also be performed by, and the apparatus disclosed herein can be implemented as, special purpose logic circuitry, such as a FPGA (field programmable gate array) or an ASIC (application specific integrated circuit). Modules and units disclosed herein can also refer to portions of the computer program and/or the processor/special circuitry that implements that functionality.

The processor 311 is any logic circuitry that responds to, processes or manipulates instructions received from the main memory unit, and can be any suitable processor for execution of a computer program. For example, the processor 311 can be a general and/or special purpose microprocessor and/or a processor of a digital computer. The CPU 315 can be any suitable processing unit known in the art. For example, the CPU 315 can be a general and/or special purpose microprocessor, such as an application-specific instruction set processor, graphics processing unit, physics processing unit, digital signal processor, image processor, coprocessor, floating-point processor, network processor, and/or any other suitable processor that can be used in a digital computing circuitry. Alternatively, or additionally, the processor can comprise at least one of a multi-core processor and a front-end processor. Generally, the processor 311 can be embodied in any suitable manner. For example, the processor 311 can be embodied as various processing means such as a microprocessor or other processing element, a coprocessor, a controller or various other computing or processing devices including integrated circuits such as, for example, an ASIC (application specific integrated circuit), an FPGA (field programmable gate array), a hardware accelerator, or the like. Additionally, or alternatively, the processor 311 can be configured to execute instructions stored in the memory 305 or otherwise accessible to the processor 311. As such, whether configured by hardware or software methods, or by a combination thereof, the processor 311 can represent an entity (e.g., physically embodied in circuitry) capable of performing operations according to embodiments disclosed herein while configured accordingly. Thus, for example, when the processor 311 is embodied as an ASIC, FPGA or the like, the processor 311 can be specifically configured hardware for conducting the operations described herein. Alternatively, as another example, when the processor 311 is embodied as an executor of software instructions, the instructions can specifically configure the processor 311 to perform the operations described herein. In many embodiments, the central processing unit 530 is provided by a microprocessor unit, e.g.: those manufactured by Intel Corporation of Mountain View, Calif; those manufactured by Motorola Corporation of Schaumburg, Ill.; the ARM processor and TEGRA system on a chip (SoC) manufactured by Nvidia of Santa Clara, Calif; the POWER7 processor, those manufactured by International Business Machines of White Plains, N.Y.; or those manufactured by Advanced Micro Devices of Sunnyvale, Calif. The processor can be configured to receive and execute instructions received from the main memory 305.

The electronic device 300 applicable to the hardware of the present invention can be based on any of these processors, or any other processor capable of operating as described herein. The central processing unit 315 may utilize instruction level parallelism, thread level parallelism, different levels of cache, and multi-core processors. A multi-core processor may include two or more processing units on a single computing component. Examples of multi-core processors include the AMD RYZEN series processors, and the INTEL CORE i5, i7, i9, X-series, H-series, U-series, and S-series processors.

The processor 311 and the CPU 315 can be configured to receive instructions and data from the main memory 305 (e.g., a read-only memory or a random-access memory or both) and execute the instructions. The instructions and other data can be stored in the main memory 305. The processor 311 and the main memory 305 can be included in or supplemented by special purpose logic circuitry. The main memory unit 305 can include one or more memory chips capable of storing data and allowing any storage location to be directly accessed by the processor 311. The main memory unit 305 may be volatile and faster than other memory in the electronic device, or can dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 305 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 305 can be based on any of the above-described memory chips, or any other available memory chips capable of operating as described herein. In the embodiment shown in FIG. 9, the processor 311 communicates with main memory 305 via a system bus 365. The computer executable instructions of the present invention may be provided using any computer-readable media that is accessible by the computing or electronic device 300. Computer-readable media may include, for example, the computer memory or storage unit 305. The computer storage media may also include, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device. In contrast, communication media may embody computer readable instructions, data structures, program modules, or other data in a modulated data signal, such as a carrier wave, or other transport mechanism. As defined herein, computer readable storage media does not include communication media. Therefore, a computer storage or memory medium should not be interpreted to be a propagating signal per se or stated another transitory in nature. The propagated signals may be present in a computer storage media, but propagated signals per se are not examples of computer storage media, which is intended to be non-transitory. Although the computer memory or storage unit 305 is shown within the computing device 300 it will be appreciated that the storage may be distributed or located remotely and accessed via a network or other communication link.

The main memory 305 can comprise an operating system 320 that is configured to implement various operating system functions. For example, the operating system 320 can be responsible for controlling access to various devices, memory management, and/or implementing various functions of the asset management system disclosed herein. Generally, the operating system 320 can be any suitable system software that can manage computer hardware and software resources and provide common services for computer programs.

The main memory 305 can also hold application software 330. For example, the main memory 305 and application software 330 can include various computer executable instructions, application software, and data structures, such as computer executable instructions and data structures that implement various aspects of the embodiments described herein. For example, the main memory 305 and application software 330 can include computer executable instructions, application software, and data structures, such as computer executable instructions and data structures that implement various aspects of the content characterization systems disclosed herein, such as processing and capture of information. Generally, the functions performed by the content characterization systems disclosed herein can be implemented in digital electronic circuitry or in computer hardware that executes software, firmware, or combinations thereof. The implementation can be as a computer program product (e.g., a computer program tangibly embodied in a non-transitory machine-readable storage device) for execution by or to control the operation of a data processing apparatus (e.g., a computer, a programmable processor, or multiple computers). Generally, the program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network, such as the Internet.

The processor 311 can further be coupled to a database or data storage 380. The data storage 380 can be configured to store information and data relating to various functions and operations of the content characterization systems disclosed herein. For example, as detailed above, the data storage 380 can store information including but not limited to captured information, multimedia, processed information, and characterized content.

A wide variety of I/O devices may be present in or connected to the electronic device 300. For example, the electronic device can include a display 370, and as previously described, the visual application unit 28 or one or more other elements of the system 10 can include the display. The display 370 can be configured to display information and instructions received from the processor 311. Further, the display 370 can generally be any suitable display available in the art, for example a Liquid Crystal Display (LCD), a light emitting diode (LED) display, digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays, or electronic papers (e-ink) displays. Furthermore, the display 370 can be a smart and/or touch sensitive display that can receive instructions from a user and forwarded the received information to the processor 311. The input devices can also include user selection devices, such as keyboards, mice, trackpads, trackballs, touchpads, touch mice, multi-touch touchpads, touch mice and the like, as well as microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. The output devices can also include video displays, graphical displays, speakers, headphones, inkjet printers, laser printers, and 3D printers.

The electronic device 300 can also include an Input/Output (I/O) interface 350 that is configured to connect the processor 311 to various interfaces via an input/output (I/O) device interface 380. The device 300 can also include a communications interface 360 that is responsible for providing the circuitry 300 with a connection to a communications network (e.g., communications network 120). Transmission and reception of data and instructions can occur over the communications network.

The electronic or computing device 300 may be designed and/or configured to perform any method, method step, or sequence of method steps in any embodiment described in this disclosure, in any order and with any degree of repetition. For instance, electronic device 300 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. The electronic device 300 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

It will thus be seen that the invention efficiently attains the objects set forth above, among those made apparent from the preceding description. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover all generic and specific features of the invention described herein, and all statements of the scope of the invention which, as a matter of language, might be said to fall therebetween.

We claim:

1. A computer-implemented patient access determination system, comprising
a processor, and
a non-transitory memory having instructions configuring the processor to:
extract health related data from a data source using an extract, transform and load technique to form extracted health data, wherein the health data includes patient encounter data, lost appointment data, and schedule data,
generate a plurality of data pipelines with the extract, transform and load technique for conveying the extracted health data,
store at least a portion of the extracted health data conveyed over one or more of the plurality of data pipelines in a data model to form stored health data, wherein the data model includes a plurality of tables for organizing and storing the extracted health data,
determine from at least the patient encounter data forming part of the stored health data a number of lost appointments that can be recovered by the healthcare facility,
apply one or more machine learning models to the stored health data to generate predictions therefrom, and
generate one or more user interfaces for displaying selected portions of the stored health data and the predictions.

2. The computer-implemented system of claim 1, wherein determine from the health data stored in the data model a number of lost appointments that can be recovered by the healthcare facility comprises configuring the processor to:
categorize the patient encounter data and the lost appointment data into a plurality of appointment categories and generate categorization data based thereon, and
determine based on the categorization data a number of recoverable appointments from the lost appointments, wherein the lost appointments were lost based on manageable reasons.

3. The computer-implemented system of claim 2, wherein the processor is further configured to determine, based on the lost appointment data in the categorization data, one or more appointment parameters associated with the lost appointments.

4. The computer-implemented system of claim 3, wherein the processor is further configured to determine a recovery factor based on the lost appointment data in the categorization data, wherein the lost appointments were lost for manageable reasons.

5. The computer-implemented system of claim 4, wherein the processor is further configured to apply the recovery factor to each category of lost appointments so as to determine the number of recoverable appointments from the lost appointments.

6. The computer-implemented system of claim 5, wherein the processor is further configured to apply a prestored reimbursement rate dynamic factor to the lost appointment data to determine a reimbursement amount based on the number of lost appointments.

7. The computer-implemented system of claim 6, wherein the processor is further configured to determine, based on the categorization data, a lost opportunity associated with the lost appointments that are lost for non-recoverable reasons.

8. The computer-implemented system of claim 7, wherein the processor is further configured to determine, based on the stored health data, an access opportunity indicative of an availability of patient appointments at a selected healthcare facility.

9. The computer-implemented system of claim 1, wherein the processor is further configured to:
categorize the lost appointment data into a plurality of different categories with a categorization unit that generates categorization data, wherein the categorization data includes lost appointment data, and
determine, based on the categorization data, a lost opportunity associated with the lost appointments that are lost for non-recoverable reasons.

10. The computer-implemented system of claim 9, wherein the plurality of different categories includes a first category for storing non-recoverable canceled appointment data from the lost appointment data and a second category for storing patient non-recoverable canceled appointment data from the lost appointment data, and wherein the processor is further configured to:
apply a recovery factor to each of the plurality of categories to determine a number of recovered appointments, and
apply a reimbursement rate to determine revenue associated with the number of recovered appointments.

11. The computer-implemented system of claim 10, wherein the processor is further configured to determine from the stored health data an access opportunity that the patient has access to appointments available at a selected healthcare facility.

12. The computer-implemented system of claim 8, wherein the processor is configured to:
train the machine learning model with the health data including lost appointment data to form a trained machine learning model, and
tune the trained machine learning model to perform one or more selected tasks by training the machine learning model on a narrower dataset of the health data and adjusting one or more tuning parameters to perform the selected task.

13. The computer-implemented system of claim 12, wherein the processor is configured to generate the plurality of data pipelines by:
generating a patient encounter standardization pipeline using the extract, transform and load technique to convey patient encounter data and to standardize the patient encounter data using a standardization technique,
generating a temporal master file creation pipeline using the extract, transform and load technique to convey one or more temporal master files based on the patient encounter data and from provider schedule data,
generating a final master file creation pipeline using the extract, transform and load technique to convey a final master file based on the temporal master files,
generating a file normalization pipeline using the extract, transform and load technique to normalize and standardize one or more files associated with the patient encounter data and provider schedule data,
generating a patient encounter final output pipeline using the extract, transform and load technique to create and convey a final patient encounter output, and
generating a provider schedule final output pipeline using the extract, transform and load technique to create and convey a final provider schedule output.

14. The computer-implemented system of claim 13, wherein the processor is configured to generate a plurality of data tables in the common data model, wherein the plurality of data tables includes
a patient encounter table for storing patient encounter data, and
a provider schedule table for storing provider schedule data associated with schedules of one or more healthcare providers associated with a healthcare facility,
and further includes four or more of:
an appointment language table for storing information associated with a language of the patient or healthcare provider,
a provider specialty table for storing information associated with a specialty of the healthcare provider in the healthcare facility,
a provider schedule-session length table for storing data associated with a length of an appointment,
a provider schedule-session per day table for storing data associated with a number of appointments per day at the healthcare facility,
appointment unavailability table for storing information associated with dates that appointments are not available in the schedule of the healthcare provider or the healthcare facility and reasons for unavailability of the appointment,
a time held table for storing information associated with time held in a schedule of the healthcare provider or the healthcare facility and one or more reasons that the time is held,
a day held table for storing information associated with a day held in a schedule of the healthcare provider or the healthcare facility and one or more reasons that the day is held,
a time unavailable table for storing information associated with a time that is unavailable in a schedule of the healthcare provider or the healthcare facility and associated appointment classification information,
a chronic diagnosis table for storing diagnostic information associated with a patient and any related healthcare code information,
an appointment type table for storing information associated with a type of appointment and date information associated with the appointment,
a last appointment table for storing information associated with a last appointment of the patient, including time since last appointment and appointment cancellation information,
an appointment schedulers table for storing information associated with a person in the healthcare facility scheduling a patient appointment including person identification information and provider specialty group information,
an appointment cancellation reason table for storing information associated with a reasons that a patient appointment was cancelled,
an encounter type table for storing information associated with a type of patient encounter,
a location type table for storing information associated with a location of the healthcare provider, and
an appointment lag table for storing information associated an appointment lag data.

15. The computer-implemented system of claim 14,
wherein the patient encounter table stores appointment data including appointment date and time, reason for appointment, appointment cancellation data, status data, appointment specific codes associated with the healthcare facility, and appointment billing information,
wherein the provider schedule table stores schedule data including provider availability data, appointment start and stop time data, appointment date data, healthcare provider classification data, and a number of available appointments at the healthcare facility, and
wherein the provider specialty table stores identification information of the healthcare provider.

16. A computer-implemented method for determining patient access to a healthcare facility, comprising
extracting health related data from a data source using an extract, transform and load technique to form extracted health data, wherein the health data includes patient encounter data, lost appointment data, and schedule data,
generating a plurality of data pipelines with the extract, transform and load technique for conveying the extracted health data,
storing at least a portion of the extracted health data conveyed over one or more of the plurality of data pipelines in a data model to form stored health data, wherein the data model includes a plurality of tables for organizing and storing the extracted health data,
determining from at least the patient encounter data forming part of the health data stored a number of lost appointments that can be recovered by the healthcare facility,
applying one or more machine learning models to the stored health data to generate predictions therefrom, and
generating one or more user interfaces for displaying selected portions of the stored health data and the predictions.

17. The computer-implemented method of claim 16, wherein the step of determining from the health data stored in the data model a number of lost appointments that can be recovered by the healthcare facility comprises
categorizing the patient encounter data and the lost appointment data into a plurality of appointment categories and generate categorization data based thereon, and
determining based on the categorization data a number of recoverable appointments from the lost appointments, wherein the lost appointments were lost based on manageable reasons.

18. The computer-implemented method of claim 17, further comprising determining, based on the lost appointment data in the categorization data, one or more appointment parameters associated with the lost appointments.

19. The computer-implemented method of claim 18, further comprising determining a recovery factor based on the lost appointment data in the categorization data, wherein the lost appointments were lost for manageable reasons.

20. The computer-implemented method of claim 19, further comprising applying the recovery factor to each category of lost appointments so as to determine the number of recoverable appointments from the lost appointments.

21. The computer-implemented method of claim 20, further comprising applying a prestored reimbursement rate dynamic factor to the lost appointment data to determine a reimbursement amount based on the number of lost appointments.

22. The computer-implemented method of claim 21, further comprising determining, based on the categorization data, a lost opportunity associated with the lost appointments that are lost for non-recoverable reasons.

23. The computer-implemented method of claim 22, further comprising determining from the stored health data an access opportunity that the patient has to appointments available at a selected healthcare facility.

24. The computer-implemented method of claim 16, further comprising
categorizing the lost appointment data into a plurality of different categories, wherein the categorization unit generates categorization data which includes lost appointment data, and
determining, based on the categorization data, a lost opportunity associated with the lost appointments that are lost for non-recoverable reasons.

25. The computer-implemented method of claim 24, wherein the plurality of different categories includes a first category for storing non-recoverable canceled appointment data from the lost appointment data and a second category for storing patient non-recoverable canceled appointment data from the lost appointment data, the method further comprising
applying a recovery factor to each of the plurality of categories to determine a number of recovered appointments, and
applying a reimbursement rate to determine revenue associated with the number of recovered appointments.

26. The computer-implemented method of claim 25, further comprising determining from the stored health data an access opportunity that the patient has to appointments available at a selected healthcare facility.

27. The computer-implemented method of claim 23, further comprising
training the machine learning model with the health data including lost appointment data to form a trained machine learning model, and
tuning the trained machine learning model to perform one or more selected tasks by training the machine learning model on a narrower dataset of the health data and adjusting one or more tuning parameters to perform the selected task.

28. The computer-implemented method of claim 27, further comprising generating the plurality of data pipelines by:
generating a patient encounter standardization pipeline using the extract, transform and load technique to convey patient encounter data and to standardize the patient encounter data using a standardization technique,
generating a temporal master file creation pipeline using the extract, transform and load technique to convey one or more temporal master files based on the patient encounter data and from provider schedule data,
generating a final master file creation pipeline using the extract, transform and load technique to convey a final master file based on the temporal master files,
generating a file normalization pipeline using the extract, transform and load technique to normalize and standardize one or more files associated with the patient encounter data and provider schedule data,
generating a patient encounter final output pipeline using the extract, transform and load technique to create and convey a final patient encounter output, and
generating a provider schedule final output pipeline using the extract, transform and load technique to create and convey a final provider schedule output.

29. The computer-implemented method of claim 28, further comprising generating a plurality of data tables in the common data model, wherein the plurality of data tables includes
a patient encounter table for storing patient encounter data, and
a provider schedule table for storing provider schedule data associated with schedules of one or more healthcare providers associated with a healthcare facility,
and further includes four or more of:
an appointment language table for storing information associated with a language of the patient or healthcare provider,
a provider specialty table for storing information associated with a specialty of the healthcare provider in the healthcare facility,
a provider schedule-session length table for storing data associated with a length of an appointment,
a provider schedule-session per day table for storing data associated with a number of appointments per day at the healthcare facility,
an appointment unavailability table for storing information associated with dates that appointments are not available in the schedule of the healthcare provider or the healthcare facility and reasons for unavailability of the appointment,
a time held table for storing information associated with time held in a schedule of the healthcare provider or the healthcare facility and one or more reasons that the time is held,
a day held table for storing information associated with a day held in a schedule of the healthcare provider or the healthcare facility and one or more reasons that the day is held,
a time unavailable table for storing information associated with a time that is unavailable in a schedule of the healthcare provider or the healthcare facility and associated appointment classification information,
a chronic diagnosis table for storing diagnostic information associated with a patient and any related healthcare code information,
an appointment type table for storing information associated with a type of appointment and date information associated with the appointment,
a last appointment table for storing information associated with a last appointment of the patient, including time since last appointment and appointment cancellation information,
an appointment schedulers table for storing information associated with a person in the healthcare facility scheduling a patient appointment including person identification information and provider specialty group information,
an appointment cancellation reason table for storing information associated with a reasons that a patient appointment was cancelled,
an encounter type table for storing information associated with a type of patient encounter,
a location type table for storing information associated with a location of the healthcare provider, and
an appointment lag table for storing information associated an appointment lag data.

30. A patient access determination system for determining patient access to a healthcare facility, comprising
a data source for providing health related data, wherein the health-related data includes patient encounter data, scheduling data, and lost appointment data,
an extraction unit for extracting the health-related data from the data source using an extract, transform and load technique to form extracted health data,
a patient encounter determination unit for receiving the extracted health data and for storing a portion of the extracted health data in a data model to form stored health data and for determining based on the lost appointment data in the stored health data a number of recoverable appointments from the lost appointment data,
a prediction unit for receiving the stored health data from the data model and applying one or more machine learning techniques to the stored health data to generate insights and predictions therefrom, and
a reporting unit for generating one or more user interfaces for displaying information associated with the insights and predictions.

31. The patient access determination system of claim 30, wherein the patient encounter determination unit comprises a demand opportunity determination unit for processing the stored health data and determining a demand opportunity based on the stored health data.

32. The patient access determination system of claim 31, wherein the demand opportunity determination unit comprises
a categorization unit for categorizing the stored health data including the lost appointment data into a plurality of categories and for generating categorization data associated therewith, and
an appointment recovery determination unit for determining based on the categorization data the number of recoverable appointments from the lost appointments, wherein the lost appointments were lost based on manageable reasons, and for generating recoverable appointment data associated therewith.

33. The patient access determination system of claim 32, wherein the demand opportunity determination unit further comprises an appointment parameter determination unit for determining, based on the lost appointment data in the categorization data, one or more appointment parameters associated with the lost appointments.

34. The patient access determination system of claim 33, wherein the appointment parameters include a length of an appointment based on a specialty of a healthcare provider and a historical appointment length.

35. The patient access determination system of claim 33, wherein the appointment parameter determination unit is configured to determine a recovery factor based on canceled appointment data forming part of the lost appointment data in the categorization data.

36. The patient access determination system of claim 35, wherein the appointment parameter determination unit is configured to apply the recovery factor to each category of lost appointments so as to determine the number of recoverable appointments from the lost appointments.

37. The patient access determination system of claim 36, wherein the appointment parameter determination unit is configured to apply a prestored reimbursement rate dynamic factor to the lost appointment data to determine a reimbursement amount based on the number of lost appointments.

38. The patient access determination system of claim 31, wherein the patient encounter determination unit further comprises a lost patients opportunity determination unit for determining a lost patients opportunity based on the lost appointment data.

39. The patient access determination system of claim 38, wherein the lost patients opportunity determination unit comprises
a categorization unit for categorizing the lost appointment data into a plurality of different categories, wherein the categorization unit generates categorization data which includes lost appointment data, and
a lost opportunity determination unit for determining, based on categorization data, the lost patients opportunity associated with the lost appointments that are lost for non-recoverable reasons.

40. The patient access determination system of claim 39, wherein the categorization unit employs a first category for storing non-recoverable canceled appointment data from the lost appointment data and a second category for storing patient non-recoverable canceled appointment data from the lost appointment data, and wherein the lost patients opportunity determination unit applies a recovery factor to each of the categories to determine the number of recovered appointments, and then the lost opportunity determination unit applies a reimbursement rate to the number of recovered appointments to determine the revenue associated with the recovered appointments.

41. The patient access determination system of claim 40, wherein the patient encounter determination unit further comprises an access opportunity determination unit for determining from the stored health data an access opportunity that the patient has to appointments available at the healthcare facility.

42. The patient access determination system of claim 31, wherein the demand opportunity determination unit further comprises an optimization opportunity determination unit for determining, based on the stored health data including appointment data, an optimization opportunity associated with optimizing an unavailable appointment time portion of the appointment data of the healthcare facility.

43. The patient access determination system of claim 42, wherein the optimization opportunity determination unit comprises
a categorization unit for categorizing an unavailable appointment time portion of the appointment data into a plurality of categories, and
an opportunity determination unit for determining a number of appointments that are recoverable from the unavailable appointment times.

44. The patient access determination system of claim 43, wherein the plurality of categories comprises
a first category for storing an appropriate unavailable appointment time data, and
a second category for storing an inappropriate unavailable appointment time category.

45. The patient access determination system of claim 44, wherein the opportunity determination unit is configured to:
identify a total recoverable appointment time that is recoverable from the unavailable appointment time,
determine a number of potential available appointments by applying a preselected appointment time length to the total recoverable appointment time,
apply a recovery factor to the number of potential available appointments to determine a number of actual available appointment time, and
apply a reimbursement rate to the number of actual available appointment times to determine revenue generated by the actual available appointment times.

46. The patient access determination system of claim 31, wherein the reporting unit generates a first user interface in response to the stored health data for displaying the appointment data, wherein the first user interface comprises a window having a first pane element disposed on a left hand side of the window and second and third vertically stacked pane elements disposed on a right hand side of the window, wherein the first pane element is configured to display lost appointment data

47. The patient access determination system of claim 46, wherein the first pane element includes a first display element having a first graphical element for displaying the lost appointment data and a table element for displaying and organizing the lost appointment data in a tabular format, and wherein the second pane element includes a second display element having a second graphical element for displaying the lost appointment data and the recoverable appointments data.

48. The patient access determination system of claim 47, wherein the third pane element includes a third display element having a third graphical element that displays the recoverable appointments by specialty practice in the healthcare facility.
